(19) United States
(12) Patent Application Publication
(10) Pub. No.: US 2026/0165360 A1
(43) Pub. Date: Jun. 18, 2026

(54) METHOD FOR ONE-STEP CO-FRACTIONATION OF BETA- AND KAPPA-CASEINS SIMULATING HUMAN MILK CASEIN COMPOSITION

(71) Applicant: Jiangnan University, Wuxi (CN)

(72) Inventors: Peng ZHOU, Wuxi (CN); Dasong LIU, Wuxi (CN); Guangyong MIAO, Wuxi (CN); Jing ZHANG, Wuxi (CN); Xiumei TAO, Wuxi (CN); Jie ZHANG, Wuxi (CN)

(21) Appl. No.: 19/536,360

(22) Filed: Feb. 11, 2026

Related U.S. Application Data
(63) Continuation of application No. PCT/CN2025/095884, filed on May 20, 2025.

(30) Foreign Application Priority Data
May 27, 2024 (CN) 2024106657720

(51) Int. Cl. A23L 33/00; A61K 9/00; A61K 9/20; A61K 9/48

(52) U.S. Cl. CPC A23L 33/40; A61K 9/0053; A61K 9/20; A61K 9/4858; A61K 2300/00

(57) ABSTRACT

The present disclosure discloses a method for one-step co-fractionation of β- and κ-caseins simulating a human milk casein composition, and belongs to the technical field of dairy product processing. The method of the present disclosure achieves co-fractionation of κ- and β-caseins by using a micellar casein concentrate (MCC) solution as a raw material, employing a selective calcium precipitation technique under alkaline or weakly alkaline conditions, and precisely regulating and combining a low-temperature step starting position and key process parameters in steps including casein-calcium complexation, low-temperature treatment, αₛ-casein precipitation, and κ- and β-casein precipitation. The method of the present disclosure simplifies a process flow, shortens a fractionation cycle, improves production efficiency, and exhibits a mild and safe process. The resulting co-fractionated product has a composition similar to human milk casein.

### TECHNICAL FIELD

The present disclosure relates to a method for one-step co-fractionation of β- and κ-caseins simulating a human milk casein composition, and belongs to the technical field of dairy product processing.

### BACKGROUND

Bovine and caprine caseins, as one of the main proteins in infant formula milk powder, have a composition different from that of human milk casein, thereby causing the infant formula milk powder to be less digestible and absorbable. The bovine casein includes αₛ₁-, αₛ₂-, β-, and κ-caseins, and mainly αₛ₁- and β-caseins, with the four components present in an approximate ratio of 40:10:36:14. The caprine casein includes αₛ₁-, αₛ₂-, β-, and κ-caseins, with the four components present in an approximate ratio of 24:18:46:12. The human milk casein includes αₛ₁-, β-, and κ-caseins, and mainly β- and κ-caseins, and does not contain αₛ₂-casein, with the three components present in a ratio of 9-12:68-70:18-21. Moreover, αₛ₁- and αₛ₂-caseins (particularly αₛ₂-casein) from bovine and caprine sources usually are highly allergenic. Therefore, to simulate the human milk casein composition, improve digestibility, and reduce allergenicity, it is necessary to fractionate components of bovine and caprine caseins.

Currently, the fractionation of the caseins mainly focuses on fractionating individual casein components. However, existing fractionation methods suffer from the deficiencies such as a complex fractionation process, a long fractionation cycle, use of toxic reagents and organic solvents, low yield and purity of components, and application of strong alkaline conditions, such that industrial scale-up production is difficult to achieve. A fractionated product is unsuitable for production of the infant formula milk powder. Under strong alkaline conditions, the casein undergoes dephosphorylation and deamidation reactions, leading to alterations in a molecular structure of the casein and formation of harmful by-products such as lysinoalanine, which are prone to causing safety concerns upon consumption. For example, in CN107840883B, bovine casein is used as a raw material, and dissolved by adding urea and β-mercaptoethanol, and then ion exchange is performed to achieve sequential fractionation of three individual caseins; in CN113461795A, methods such as ethanol dissolution and acid precipitation are used to achieve sequential fractionation of three individual caseins; in CN112931616A, a low-temperature membrane filtration method is used to achieve co-fractionation of β-casein and whey protein; and in CN116355073A, a calcium precipitation method is used under pH 12 conditions to achieve fractionation of κ-casein. When these individual caseins are used in the production of the infant formula milk powder, compatible co-formulation is still required to simulate the human milk casein composition, resulting in a complex, time-consuming, and costly overall process.

κ- and β-caseins are the two main caseins in human milk. Using a one-step method to co-fractionate κ- and β-caseins from bovine and caprine caseins may directly achieve simulation of the human milk casein composition, shorten a fractionation process route and a fractionation cycle, reduce costs, and further decrease a content of αₛ-casein serving as a major allergenic protein. When used in the production of the infant formula milk powder, no further compatible co-formulation with other caseins is required. However, κ-casein is a glycosylated protein with strong hydrophilicity and a low degree of phosphorylation; β-casein has strong hydrophobicity and a high degree of phosphorylation; and αₛ-casein exhibits moderate hydrophilicity-hydrophobicity and an extremely high degree of phosphorylation. Evidently, κ-casein, β-casein, and αₛ-casein differ significantly in properties, and are ranked differently in different characteristics, such that the co-fractionation of κ- and β-caseins becomes a challenging problem in the field. The co-fractionation requires simultaneously achieving a high extraction rate and high purity for κ-casein and β-casein that exhibit the greatest differences in properties. Design of a fractionation solution and selection of conditions differ from those for fractionation of any single casein. The requirements for co-fractionating κ- and β-caseins are more stringent and challenging than those for the fractionation of any single casein, and even more challenging than those for the co-fractionation of αₛ- and β-caseins. Currently, no relevant reports have been published.

### SUMMARY

### Technical Problems

Fractionation of individual caseins from animal milk suffers from deficiencies such as a complex fractionation process, a long fractionation cycle, use of toxic reagents and organic solvents, low yield and purity of components, and formation of harmful substances due to application of strong alkaline conditions. Furthermore, use of the individual caseins in production of infant formula milk powder requires compatible co-formulation, thereby leading to a limitation in industrial production and applications.

The significant differences among κ-casein, β-casein, and αₛ-casein in properties, such as hydrophilicity and hydrophobicity, a degree of phosphorylation, and a degree of glycosylation, cause the co-fractionation of κ- and β-caseins to become a challenging problem in the field.

### Technical Solution

To solve the above problems, the present disclosure achieves co-fractionation of κ- and β-caseins by using a micellar casein concentrate (MCC) solution as a raw material, employing a selective calcium precipitation technique under alkaline or weakly alkaline conditions, and precisely regulating and combining a low-temperature step starting position and key process parameters in steps including casein-calcium complexation, low-temperature treatment, αₛ-casein precipitation, and κ- and β-casein precipitation. A method of the present disclosure simplifies a process flow, shortens a fractionation cycle, improves production efficiency, and exhibits a mild and safe process. The resulting co-fractionated product has a composition similar to human milk casein, and both κ- and β-caseins are obtained with high yield.

A first objective of the present disclosure is to provide a method for co-fractionation of κ- and β-caseins from casein micelles, including the following steps:
(1) co-fractionation:
option 1: at 23-27° C., adjusting a pH of a casein solution having a mass concentration of 1-7% to 11.0; subsequently adding a calcium chloride solution until a concentration of added calcium ions reaches 5-65 mM; equilibrating a resulting solution at 10-40° C. for 0-90 minutes to obtain a casein-calcium complexed solution; then subjecting the casein-calcium complexed solution to low-temperature treatment at 0-12° C. for 0-60 minutes, adjusting a pH of the complexed solution to 4.6-5.6 for acid precipitation, and equilibrating a resulting solution at 0-12° C. for 0-24 hours; and performing centrifugation to obtain a precipitate enriched with αₛ-casein and a supernatant enriched with κ- and β-caseins;
option 2: at 23-27° C., adjusting a pH of a casein solution having a mass concentration of 2-4% to 4.3, heating the solution to 40-50° C., and equilibrating the solution for 20-40 minutes; then performing centrifugation after cooling to 23-27° C. to obtain a precipitate; collecting the precipitate, placing the precipitate in ultrapure water, and adjusting a pH of the same to 6.5-9.0 at 23-27° C., followed by equilibration at 23-27° C. for 0-75 minutes to obtain a reconstituted casein solution, where a casein mass concentration is 2-4%; adding a calcium chloride solution to the casein solution until a concentration of added calcium ions reaches 30-70 mM, and equilibrating a resulting solution at 23-27° C. for 50-70 minutes; adjusting a pH of the resulting solution to 5.0 for acid precipitation to obtain a solution, equilibrating the solution at 23-27° C. for 0-150 minutes, and then subjecting the resulting solution to low-temperature treatment at 0-12° C. for 7-9 hours; and performing centrifugation to obtain a precipitate enriched with αₛ-casein and a supernatant enriched with κ- and β-caseins; or
option 3: adjusting a pH of a casein solution having a mass concentration of 2-4% to 5.0 for acid precipitation, equilibrating a resulting solution at 23-27° C. for 80-100 minutes, further equilibrating the resulting solution at 1-4° C. for 7-9 hours, and performing centrifugation to obtain a precipitate enriched with αₛ-casein and a supernatant enriched with κ- and β-caseins; and
(2) collecting the supernatant enriched with κ- and β-caseins, adjusting a pH of the supernatant to 3.0-5.2, allowing precipitation at 15-55° C. for 0-60 min, and performing centrifugation to obtain a precipitate enriched with κ- and β-caseins.

In an embodiment of the present disclosure, in the co-fractionation option 1 of the step (1), the mass concentration of the casein solution is preferably 2-4%, and further preferably 3%.

In an embodiment of the present disclosure, in the co-fractionation option 1 of the step (1), the casein solution is obtained by reconstituting MCC powder in water or by diluting a reconstituted MCC solution.

In an embodiment of the present disclosure, in the co-fractionation option 1 of the step (1), the pH is adjusted to 11.0 with a 2 M NaOH solution.

In an embodiment of the present disclosure, in the co-fractionation option 1 of the step (1), the concentration of added calcium ions is 35 mM; the equilibration temperature after the addition of the calcium chloride is 10-25° C., and further 20° C.; and the equilibration duration is at least 60 minutes, preferably 60-90 minutes, and further preferably 60-75 minutes.

In an embodiment of the present disclosure, in the co-fractionation option 1 of the step (1), the temperature of the low-temperature treatment is 0-4° C., and the duration is at least 10 minutes; preferably, the temperature is 4° C., and the duration is 10-50 minutes; and more preferably, the duration is 10 minutes.

In an embodiment of the present disclosure, in the co-fractionation option 1 of the step (1), the pH is adjusted to 4.6-5.6 with a 4 M acetic acid solution, a 4 M hydrochloric acid solution, a 4 M lactic acid solution, or a 4 M citric acid solution; and preferably, the 4 M acetic acid solution is used.

In an embodiment of the present disclosure, in the co-fractionation option 1 of the step (1), the acid precipitation is performed at pH 4.9-5.1, and preferably pH 5.0; and the equilibration duration after the acid precipitation is 8-24 hours, and preferably 8 hours.

In an embodiment of the present disclosure, in the co-fractionation option 1 of the step (1), the centrifugation is performed at 3.5-4.5° C. and 2,000-10,000 g for 5-15 minutes; and the equilibration is an equilibration under stirring at a speed of 200-500 rpm.

In an embodiment of the present disclosure, in the co-fractionation option 2 of the step (1), the casein solution is prepared by reconstituting MCC powder in water or by diluting a reconstituted MCC solution.

In an embodiment of the present disclosure, in the co-fractionation option 2 of the step (1), the pH is adjusted to 4.3 with a 2 M HCl solution.

In an embodiment of the present disclosure, in the co-fractionation option 2 of the step (1), the centrifugation after cooling to 23-27° C. is performed at 2,000-10,000 g for 5-15 minutes.

In an embodiment of the present disclosure, in the co-fractionation option 2 of the step (1), the pH is adjusted to 6.5-9.0 with a 2 M NaOH solution.

In an embodiment of the present disclosure, in the co-fractionation option 2 of the step (1), a pH of the reconstituted casein solution is 7.5; the equilibration duration of the reconstituted casein solution is at least 60 minutes, preferably 60-75 minutes, and further preferably 60 minutes.

In an embodiment of the present disclosure, in the co-fractionation option 2 of the step (1), the concentration of added calcium ions is 45-70 mM, and preferably 65-70 mM; and the equilibration temperature after the addition of the calcium chloride is 25° C.

In an embodiment of the present disclosure, in the co-fractionation option 2 of the step (1), the pH is adjusted to 5.0 with a 4 M acetic acid solution; and the equilibration temperature after the acid precipitation is 23-27° C., and preferably 27° C.

In an embodiment of the present disclosure, in the co-fractionation option 2 of the step (1), the equilibration duration after the acid precipitation is at least 90 minutes, preferably 90-150 minutes, and further preferably 90-120 minutes.

In an embodiment of the present disclosure, in the co-fractionation option 2 of the step (1), the temperature of the low-temperature treatment is 0-2° C., and preferably 2° C.; the duration of the low-temperature treatment is 7-9 hours, and preferably 9 hours; and the centrifugation after the 7-9-hour low-temperature treatment at 0-12° C. is performed at 0-4° C. and 2,000-10,000 g for 5-15 minutes.

In an embodiment of the present disclosure, in the co-fractionation option 2 of the step (1), the equilibration is an equilibration under stirring at a speed of 200-500 rpm.

In an embodiment of the present disclosure, in the co-fractionation option 2 of the step (1), the mass concentration of the casein solution is 2-4%, and preferably 4%.

In an embodiment of the present disclosure, in the co-fractionation option 3 of the step (1), the casein solution is obtained by reconstituting MCC powder in water or by diluting a reconstituted MCC solution.

In an embodiment of the present disclosure, in the co-fractionation option 3 of the step (1), the pH is adjusted to 5.0 with a 4 M acetic acid solution, a 4 M lactic acid solution, or a 4 M citric acid solution; and preferably, the 4 M acetic acid solution is used.

In an embodiment of the present disclosure, in the co-fractionation option 3 of the step (1), the equilibration is an equilibration under stirring at a speed of 200-500 rpm.

In an embodiment of the present disclosure, in the co-fractionation option 3 of the step (1), the centrifugation is performed at 0-4° C. and 2,000-10,000 g for 5-15 minutes.

In an embodiment of the present disclosure, in the step (2), the pH is 3.4-3.8, and preferably 3.6.

In an embodiment of the present disclosure, in the step (2), the pH is adjusted to 3.0-5.2 with a 2 M hydrochloric acid solution.

In an embodiment of the present disclosure, in the step (2), the precipitation temperature is 45-55° C., and preferably 50° C.; and the precipitation duration is at least 20 minutes, preferably 20-60 minutes, and further preferably 20 minutes.

In an embodiment of the present disclosure, the MCC used for preparing the casein solution in the step (1) is a micellar casein concentrate, or referred to as a casein micelle concentrate derived from animal milk.

Specifically, the MCC is derived from animal milk such as bovine milk; and the MCC may be obtained commercially, or prepared by membrane filtration separation, or obtained by diluting a retentate from membrane filtration with water.

In an embodiment of the present disclosure, the MCC is prepared by a method as follows:
sterilizing skimmed milk through a ceramic membrane with a pore size of 1.4 μm, removing whey protein through a ceramic membrane with a pore size of 100 nm, with a volume concentration factor of 4, and performing water diafiltration three times, and collecting and lyophilizing a resulting retentate to obtain MCC powder.

A second objective of the present disclosure is κ- and β-caseins prepared through the above method of the present disclosure.

A third objective of the present disclosure is use of the method or the κ- and β-caseins of the present disclosure in food products.

In an embodiment of the present disclosure, the use involves infant and young child food products, and particularly preparation of infant and young child formula milk powder.

### Beneficial Effects

(1) The co-fractionation option 1 for κ- and β-caseins according to the present disclosure achieves the yield and purity of κ-casein reaching 82.1% and 15.3%, and the yield and purity of β-casein reaching 96.1% and 73.8% by using the MCC prepared through membrane filtration as the raw material, by controlling precise regulation and combination of different low-temperature starting positions and key process parameters in main steps including casein-calcium complexation, low-temperature treatment, and αₛ-casein precipitation, and further through the control and combined effect of process parameters in steps including κ- and β-casein precipitation.

(2) The co-fractionation option 2 for κ- and β-caseins according to the present disclosure achieves the yield and purity of κ-casein reaching 73.5% and 15.2%, and the yield and purity of β-casein reaching 97.7% and 67.7%, by using the MCC as the raw material, by controlling the precise regulation and combination of weak alkaline re-dissolution conditions, different low-temperature starting positions, and key process parameters in main steps including casein-calcium complexation, low-temperature treatment, and αₛ-casein precipitation, and further through the control and combined effect of process parameters in steps including κ- and β-casein precipitation; still further, achieves the yield and purity of κ-casein reaching 85.7% and 18.3%, and the yield and purity of β-casein reaching 98.7% and 69.6% by controlling the equilibration temperature after the acid precipitation, the low-temperature equilibration duration, and the casein mass concentration; yet further, achieves the yield and purity of κ-casein reaching 90.2% and 20.9%, and the yield and purity of β-casein reaching 99.1% and 70.2% after increasing the casein concentration, by controlling the concentration of added calcium ions to further precipitate and remove αₛ₁-casein and αₛ₂-casein, the latter of which is completely removed.

(3) The co-fractionation option 3 for κ- and β-caseins according to the present disclosure achieves the yield and purity of κ-casein reaching 43.9% and 14.0%, and the yield and purity of β-casein reaching 76.5% and 81.4% by using the MCC as the raw material, by controlling the regulation and combination of key process parameters in main steps including αₛ-casein precipitation and low-temperature treatment, and further through the control and combined effect of process parameters in the κ- and β-casein precipitation step.

(4) The present disclosure provides a method for one-step co-fractionation of κ- and β-caseins. Compared with a multi-step method involving sequential fractionation of individual caseins, the method simplifies a process flow, shortens a fractionation cycle, and achieves high yield and purity for both κ- and β-caseins through a specific combination and combined effect of key process parameters in various steps. Moreover, the method enables the co-fractionation of κ- and β-caseins at a relatively high casein concentration, and improves production efficiency, such that the method is suitable for industrial scale-up production.

(5) The purities of κ- and β-caseins in the co-fractionated product obtained by the method of the present disclosure are very close to the purities of these two caseins in human milk, and a content of αₛ-casein serving as a major allergenic protein is low. Therefore, the co-fractionated product may be directly used in production of infant formula milk powder to simulate the human milk casein composition, without the need for compatible co-formulation with other caseins to adjust the proportions of various caseins. The production cost is relatively low, and the co-fractionated product serves as a novel casein ingredient to promote iterative upgrading of the infant formula milk powder.

(6) Compared with conventional strong alkaline process conditions, the co-fractionated product obtained under the weak alkaline process conditions (pH 7.5) of the present disclosure exhibits significantly higher yields and purities for both κ-casein and β-casein. The purities of κ-casein and β-casein are closer to the purities of these two caseins in human milk, and a content of αₛ₂-casein is extremely low, thereby achieving a casein composition closer to human milk. It is even possible to achieve the purities of κ-casein and β-casein similar to the purities of these two caseins in the human milk, and no αₛ₂-casein contained, such that the casein composition of the fractionated product is similar to the casein composition of the human milk.

(7) In the method of the present disclosure, no toxic reagents or organic solvents are used. Moreover, compared with a strong alkaline process, the extents of dephosphorylation and deamidation reactions occurring in the weak alkaline process are minimal, and a content of a harmful substance lysinoalanine generated is also extremely low, indicating the mildness and safety of the weak alkaline process. The resulting co-fractionated product is suitable for production of food products with high requirements for raw materials, such as infant formula milk powder.

### BRIEF DESCRIPTION OF THE DRAWINGS

FIG. 1 is a process flowchart of a co-fractionation option 1 for κ- and β-caseins.

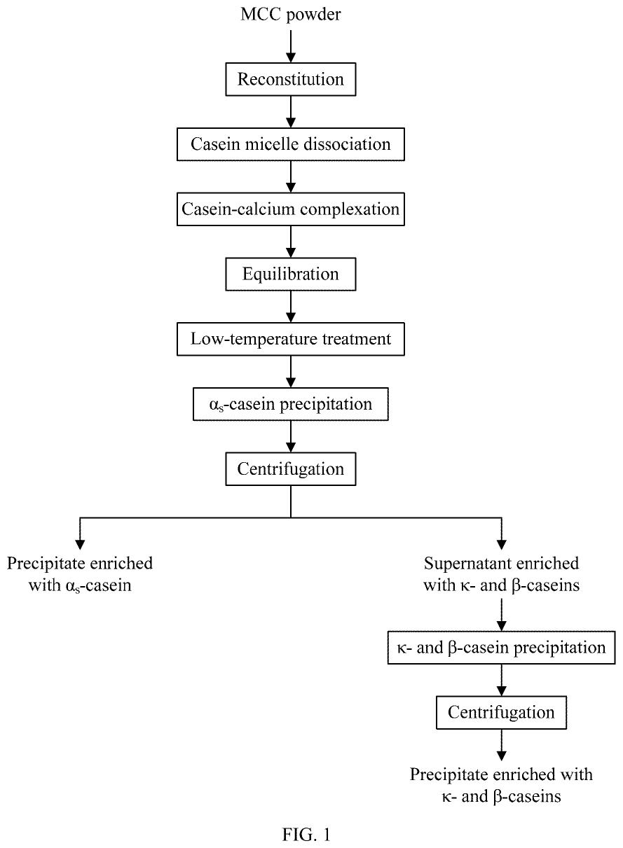

FIG. 2 is a process flowchart of a co-fractionation option 2 for κ- and β-caseins.

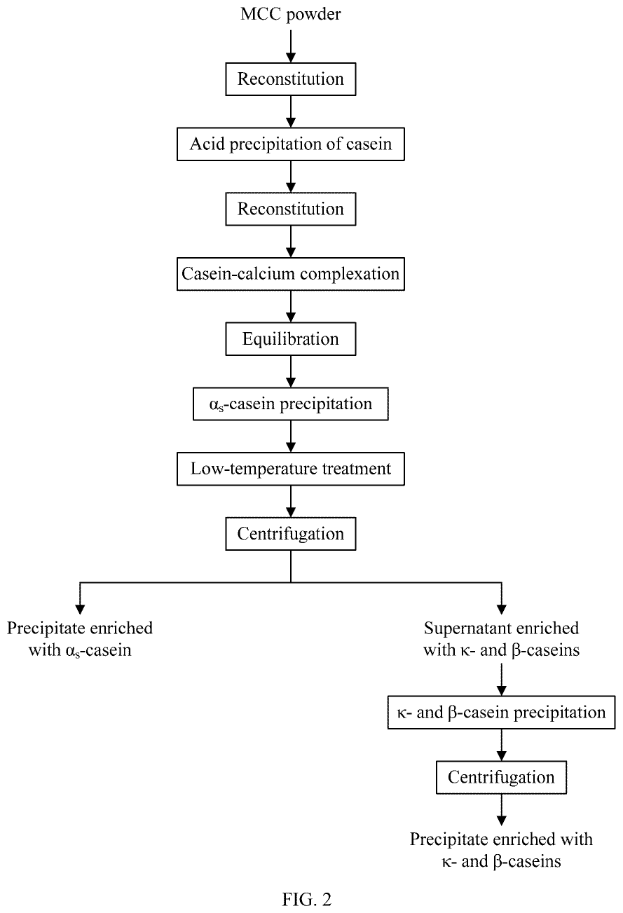

FIG. 3 is a process flowchart of a co-fractionation option 3 for κ- and β-caseins.

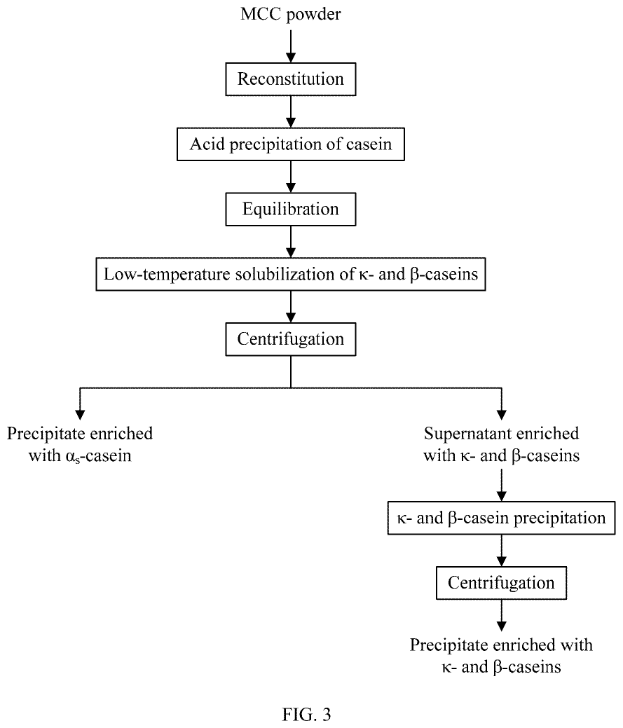

FIG. 4A is a reversed-phase high performance liquid chromatogram of MCC in Example 1.

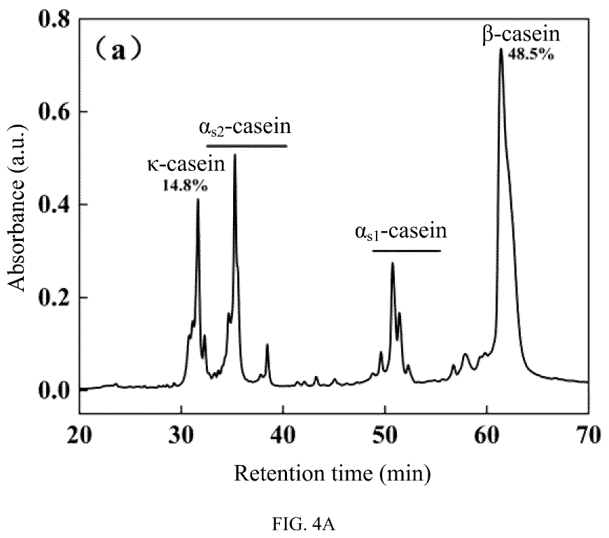

FIG. 4B is a reversed-phase high performance liquid chromatogram of co-fractionated κ- and β-caseins in Example 1.

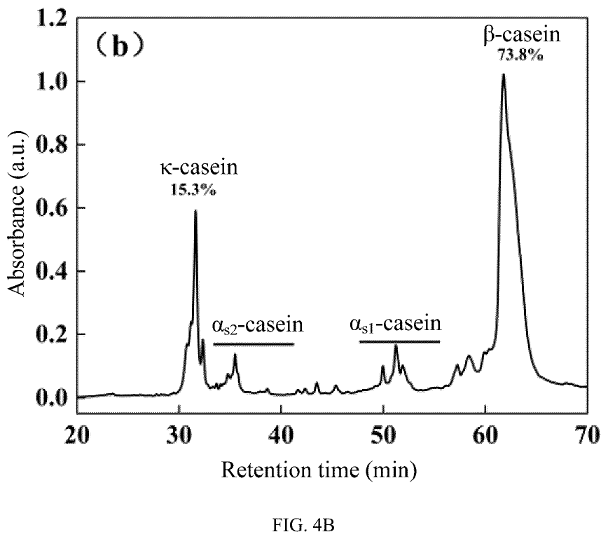

FIG. 5 is a reversed-phase high performance liquid chromatogram of co-fractionated κ- and β-caseins in Example 14.

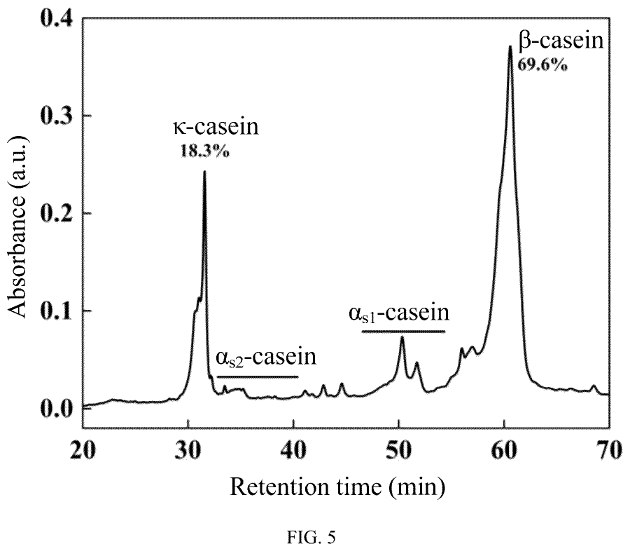

### DETAILED DESCRIPTIONS OF THE EMBODIMENTS

Preferred embodiments of the present disclosure will be described below. It should be understood that these embodiments are provided to better explain the present disclosure and are not intended to limit the present disclosure.

### Test Methods:

### 1. Reversed-Phase High-Performance Liquid Chromatography (RP-HPLC) Analysis

The casein composition is determined by an e2695 high-performance liquid chromatograph (Waters Corp., Milford, MA, USA) equipped with an XBridge BEH C18 column (250 mm×4.6 mm). A detection wavelength is 220 nm. Chromatographic peaks are integrated by Empower software.

### 2. Definitions of Extraction Rate, Purity, Precipitation Rate, and Yield for κ- and β-Caseins

Extraction rate: refers to a ratio of a peak area of κ-casein or β-casein in a supernatant enriched with κ- and β-caseins to a peak area of κ-casein or β-casein in an initial reconstituted MCC solution.

Purity: refers to a ratio of a peak area of κ-casein or β-casein in a precipitate or supernatant enriched with κ- and β-caseins to a peak area of all caseins.

Precipitation rate: is calculated as 1 minus a proportion of non-precipitated κ-casein or β-casein, where the proportion of non-precipitated κ-casein or β-casein is defined as a ratio of a peak area of κ-casein or β-casein in a supernatant depleted in κ- and β-caseins to a peak area of κ-casein or β-casein in a supernatant enriched with κ- and β-caseins.

Yield: refers to a product of an extraction rate and a precipitation rate.

### 3. Phosphate Group Removal, Amide Group Removal, and Lysinoalanine Content Analysis

A solution obtained prior to centrifugation to co-fractionate κ- and β-caseins is taken, a 30% trichloroacetic acid solution is added, and a supernatant is collected. A phosphate content is determined through a phosphomolybdic acid colorimetric method, and a free ammonia content is determined through a phenol-sodium hypochlorite colorimetric method. Caseins are taken and hydrolyzed with 6 M HCl, and a lysinoalanine content is determined by liquid chromatography-mass spectrometry (LC-MS).

In the examples, “%” involved refers to a mass percentage, unless otherwise specified; and a solution involved refers to an aqueous solution using water as a solvent, unless otherwise specified.

### Example 1: Co-Fractionation Option 1 for κ- and β-Caseins in MCC

A method for co-fractionation of κ- and β-caseins includes the following steps:

(1) MCC powder was taken and reconstituted in ultrapure water to obtain a reconstituted casein solution having a casein mass concentration of 3%.

(2) At 25° C., a 2 M NaOH solution was added to the reconstituted casein solution to adjust a pH to 11.0.

(3) A 2 M calcium chloride solution was added until a concentration of added calcium ions reached 35 mM, and a resulting solution was equilibrated under stirring (300 rpm) for 60 minutes after being cooled to 20° C. to obtain a casein-calcium complexed solution.

(4) The casein-calcium complexed solution was subjected to low-temperature treatment under stirring (300 rpm) for 10 minutes after being cooled to 4° C.

(5) A 4 M acetic acid solution was added to adjust a pH of the complexed solution to 5.0 for acid precipitation, and a resulting solution was equilibrated under stirring (300 rpm) at 4° C. for 8 hours.

(6) Centrifugation was performed at 4° C. and 3,000 g for 10 minutes to obtain a precipitate enriched with αₛ-casein and a supernatant enriched with κ- and β-caseins.

(7) The supernatant enriched with κ- and β-caseins was collected, a 2 M hydrochloric acid solution was added at 25° C. to adjust a pH of the supernatant to 3.6, and a resulting solution was equilibrated under stirring (300 rpm) for 20 minutes after being heated to 50° C., and centrifuged at 10,000 g for 10 minutes after being cooled to 25° C. to obtain a precipitate enriched with κ- and β-caseins and a supernatant depleted in κ- and β-caseins.

### Comparative Example 1: Extraction of κ- and β-Caseins by Two-Step pH Lowering Method

Extraction of κ- and β-caseins by a two-step pH lowering method includes the following steps:

(1) MCC powder was taken and reconstituted in ultrapure water to obtain a reconstituted casein solution having a casein mass concentration of 3%.

(2) At 25° C., a 2 M NaOH solution was added to the reconstituted casein solution to adjust a pH of the reconstituted casein solution to 11.0.

(3) At 25° C., a 2 M calcium chloride solution was added until a concentration of added calcium ions reached 35 mM, and a resulting solution was equilibrated under stirring (300 rpm) for 60 minutes to obtain a casein-calcium complexed solution.

(4) A 2 M acetic acid solution was added to adjust a pH of the complexed solution to 7.0, and a resulting solution was equilibrated under stirring (300 rpm) at 25° C. for 60 minutes.

(5) The equilibrated solution was subjected to low-temperature treatment under stirring (300 rpm) at 4° C. for 2 hours, a 2 M acetic acid solution was added to adjust a pH of the treated solution to 5.0, and a resulting solution was equilibrated again under stirring (300 rpm) at 4° C. for 12 hours.

The steps (6) and (7) were the same as the steps (6) and (7) in Example 1.

The yields and purities of κ-casein and β-casein obtained in step (7) in Example 1 and Comparative Example 1 are shown in Table 1. As can be seen from Table 1, the method of Comparative Example 1 significantly affects the yields of κ- and β-caseins in the co-fractionation process; and the yield of β-casein in Comparative Example 1 is lower than the yield of β-casein in Example 1, and the yield of κ-casein in Comparative Example 1 is significantly lower than the yield of κ-casein in Example 1. Moreover, the process flow of Comparative Example 1 is relatively more complex. Therefore, the solution of Example 1 is preferred for co-fractionation of κ- and β-caseins.

### Comparative Example 2: Extraction of κ- and β-Caseins by Non-pH-Lowering Method

Extraction of κ- and β-caseins by a non-pH-lowering method includes the following steps:

(1) MCC powder was taken and reconstituted in ultrapure water to obtain a reconstituted casein solution having a casein mass concentration of 3%.

(2) At 25° C., a 2 M NaOH solution was added to the reconstituted casein solution to adjust a pH of the reconstituted casein solution to 11.0.

(3) A 2 M calcium chloride solution was added until a concentration of added calcium ions reached 35 mM, and a resulting solution was equilibrated under stirring (300 rpm) for 60 minutes after being cooled to 20° C. to obtain a casein-calcium complexed solution.

(4) The casein-calcium complexed solution was subjected to low-temperature equilibration under stirring (300 rpm) at 4° C. for 12 hours after being cooled to 4° C.

The steps (5) and (6) were the same as the steps (6) and (7) in Example 1.

The yields and purities of κ-casein and β-casein obtained in Example 1 and Comparative Example 2 are shown in Table 1. As can be seen from Table 1, the method of Comparative Example 2 significantly affects the purities of κ- and β-caseins in the co-fractionation process, and the purities of κ- and β-caseins are lower than the purities of κ- and β-caseins in Example 1. This result is attributed to the excessively high pH in the low-temperature step in Comparative Example 2, which is unfavorable for the precipitation and removal of αₛ-casein.

TABLE 1
Co-fractionation results of κ- and β-caseins in Example
1, Comparative Example 1, and Comparative Example 2

| | κ-casein | | β-casein | |
|---|---|---|---|---|
| Example | Yield (%) | Purity (%) | Yield (%) | Purity (%) |
| Example 1 | 82.1 ± 0.5 | 15.3 ± 0.2 | 96.1 ± 0.9 | 73.8 ± 0.4 |
| Comparative Example 1 | 43.4 ± 1.0 | 14.7 ± 0.4 | 76.7 ± 0.9 | 66.9 ± 0.7 |
| Comparative Example 2 | 83.2 ± 1.1 | 12.5 ± 0.5 | 98.1 ± 1.2 | 47.2 ± 0.8 |

### Example 2: Co-Fractionation Option 1: Effect of Different Low-Temperature Starting Positions

Control of different low-temperature starting positions:

(1) Before casein micelle dissociation:

The step (4) in Example 1 was adjusted to precede the step (2), and the temperature in the subsequent steps was adjusted to 4° C. Other conditions remained consistent with those in Example 1.

(2) Before casein-calcium complexation:

The step (4) in Example 1 was adjusted to precede the step (3), and the temperature in the subsequent steps was adjusted to 4° C. Other conditions remained consistent with those in Example 1.

(3) After casein-calcium complexation:

The step (4) in Example 1 was adjusted to follow “a calcium chloride solution was added until a concentration of added calcium ions reached 35 mM” in the step (3), the adjusted treatment involved that a resulting solution was equilibrated under stirring (300 rpm) for 60 minutes after being cooled to 4° C., and the temperature in the subsequent steps was adjusted to 4° C. Other conditions remained consistent with those in Example 1.

(4) Before αₛ-casein precipitation:

Conditions remained consistent with those in Example 1.

(5) After αₛ-casein precipitation:

The step (4) in Example 1 was adjusted to follow “a 4 M acetic acid solution was added to adjust a pH of the complexed solution to 5.0 for acid precipitation” in the step (5). Other conditions remained consistent with those in Example 1.

In the low-temperature starting position regulation solutions, the extraction rates and purities of κ-casein and β-casein obtained in the step (6) are shown in Table 2. As can be seen from Table 2, as the low-temperature starting position is delayed in the process, the extraction rates of both κ-casein and β-casein show a decreasing trend. Specifically, when the low-temperature step starting position occurs after the αₛ-casein precipitation, the extraction rates of two caseins decrease significantly; and as the low-temperature starting position is delayed, the purities of both κ-casein and β-casein increase, where when the low-temperature step starting position occurs after the αₛ-casein precipitation, the purities of two caseins change minimally. Delaying the low-temperature step starting position enhances the calcium-binding capacity of the caseins, which is beneficial for selective fractionation of caseins. Therefore, the low-temperature starting position occurring before the αₛ-casein precipitation is preferred for the co-fractionation of κ- and β-caseins.

TABLE 2
Co-fractionation results in low-temperature starting
position regulation solutions in Example 2

| | κ-casein | | β-casein | |
|---|---|---|---|---|
| Low-temperature | Extraction | Purity | Extraction | Purity |
| starting position | rate (%) | (%) | rate (%) | (%) |
| Before casein micelle | 97.9 ± 1.1 | 14.0 ± 0.3 | 98.1 ± 1.3 | 57.1 ± 0.8 |
| dissociation | | | | |
| Before casein-calcium | 93.7 ± 0.8 | 15.1 ± 0.2 | 97.3 ± 1.7 | 64.0 ± 0.7 |
| complexation | | | | |
| After casein-calcium | 89.5 ± 0.8 | 16.4 ± 1.0 | 96.6 ± 0.9 | 70.7 ± 0.5 |
| complexation | | | | |
| Before αₛ-casein | 87.6 ± 1.0 | 17.8 ± 0.4 | 96.2 ± 0.7 | 74.1 ± 0.8 |
| precipitation (Example 1) | | | | |
| After αₛ-casein | 75.4 ± 0.9 | 18.0 ± 0.7 | 83.9 ± 1.1 | 75.5 ± 0.6 |
| precipitation | | | | |

### Example 3: Co-Fractionation Option 1: Effects of Different Concentrations of Added Calcium Ions, Casein Mass Concentrations, Equilibration Temperatures, and Equilibration Durations

(1) Regulation of different concentrations of added calcium ions: The concentration of added calcium ions in the step (3) of Example 1 was adjusted to 5, 15, 25, 45, 55, and 65 mM, respectively. All other steps remained consistent with those in Example 1.

In the regulation solutions for the concentration of added calcium ions, the extraction rates and purities of κ-casein and β-casein obtained in the step (6) are shown in Table 3. As can be seen from Table 3, when the concentration of added calcium ions falls within a range of 5-35 mM, the extraction rates of both κ-casein and β-casein are decreased; and when the concentration of added calcium ions falls within a range of 35-65 mM, the extraction rates of both κ-casein and β-casein are increased. The purities of both κ-casein and β-casein show an increasing trend when the concentration of added calcium ions falls within the range of 5-35 mM, and a decreasing trend when the concentration of added calcium ions falls within the range of 35-65 mM. Therefore, a concentration of added calcium ions of 35 mM is preferred for the co-fractionation of κ- and β-caseins, and high-purity κ-casein and β-casein are obtained.

TABLE 3
Co-fractionation results in regulation solutions for
the concentration of added calcium ions in Example 3

| Calcium ion | κ-casein | | β-casein | |
|---|---|---|---|---|
| concentration | Extraction | Purity | Extraction | Purity |
| (mM) | rate (%) | (%) | rate (%) | (%) |
| 5 | 91.9 ± 1.1 | 15.8 ± 0.4 | 98.4 ± 0.9 | 67.3 ± 0.5 |
| 15 | 91.4 ± 0.8 | 16.5 ± 0.6 | 97.9 ± 1.0 | 67.5 ± 1.1 |
| 25 | 88.9 ± 1.0 | 17.6 ± 0.7 | 96.6 ± 1.0 | 72.0 ± 0.9 |
| 35 (Example 1) | 87.6 ± 1.0 | 17.8 ± 0.4 | 96.2 ± 0.7 | 74.1 ± 0.8 |
| 45 | 88.9 ± 0.9 | 17.5 ± 0.9 | 96.3 ± 0.5 | 72.4 ± 1.3 |
| 55 | 90.9 ± 0.5 | 17.4 ± 0.8 | 97.0 ± 0.9 | 71.9 ± 0.7 |
| 65 | 91.0 ± 0.7 | 17.0 ± 1.0 | 97.4 ± 1.0 | 70.8 ± 0.6 |

(2) Regulation of different casein mass concentrations: The casein mass concentration in the step (1) of Example 1 was adjusted to 1, 2, 4, 5, 6, and 7%, respectively. All other steps remained consistent with those in Example 1.

In the casein mass concentration regulation solutions, the extraction rates and purities of κ-casein and β-casein obtained in the step (6) are shown in Table 4. As can be seen from Table 4, as the casein mass concentration is increased from 1% to 3%, the extraction rates of both κ-casein and β-casein are decreased slightly; and when the casein mass concentration is further increased to 7%, the extraction rates of both κ-casein and β-casein are decreased significantly. As the casein mass concentration is increased from 1% to 3%, the purities of both κ-casein and β-casein are increased slightly; and when the casein mass concentration is further increased to 7%, the purity of κ-casein is further increased, while the purity of β-casein is decreased significantly. The higher the casein mass concentration, the higher the solution viscosity, which hinders the uniform dispersion of added calcium ions and an acidifying agent, and causes casein aggregation in localized regions. Moreover, higher viscosity is also unfavorable for the precipitation of caseins. These factors adversely affect the fractionation of caseins. It is also observed from the experiment that when the casein mass concentration is >8%, gel formation occurs in the solution in the fractionation process, thereby hindering subsequent treatment. Therefore, a casein mass concentration of 3% is preferred for the co-fractionation of κ- and β-caseins.

TABLE 4
Co-fractionation results in casein mass concentration
regulation solutions in Example 3

| | κ-casein | | β-casein | |
|---|---|---|---|---|
| Casein mass | Extraction | Purity | Extraction | Purity |
| concentration (%) | rate (%) | (%) | rate (%) | (%) |
| 1 | 88.4 ± 0.8 | 15.2 ± 0.7 | 98.3 ± 0.9 | 73.8 ± 0.5 |
| 2 | 88.1 ± 1.2 | 16.6 ± 1.0 | 97.9 ± 1.4 | 73.9 ± 1.0 |
| 3 (Example 1) | 87.6 ± 1.0 | 17.8 ± 0.4 | 96.2 ± 0.7 | 74.1 ± 0.8 |
| 4 | 86.3 ± 0.5 | 18.9 ± 0.9 | 89.5 ± 1.0 | 72.1 ± 0.8 |
| 5 | 82.5 ± 1.4 | 20.3 ± 1.0 | 77.7 ± 1.1 | 71.2 ± 0.8 |
| 6 | 80.5 ± 0.6 | 20.6 ± 0.6 | 73.9 ± 0.5 | 70.6 ± 0.9 |
| 7 | 73.5 ± 0.8 | 22.5 ± 0.8 | 62.9 ± 0.9 | 67.7 ± 0.6 |

(3) Regulation of different equilibration temperatures: The temperature of the solution after the addition of the calcium chloride in the step (3) of Example 1 was adjusted to 10, 15, 25, 30, 35, and 40° C., respectively. All other steps remained consistent with those in Example 1.

In the equilibration temperature regulation solutions, the extraction rates and purities of κ-casein and β-casein obtained in the step (6) are shown in Table 5. As can be seen from Table 5, the extraction rates of both κ-casein and β-casein are decreased slightly within a range of 10-20° C. and decreased significantly within a range of 20-40° C. Within a range of 10-35° C., the purities of both κ-casein and β-casein exhibit an increasing trend; within a range of 15-20° C., the purity of β-casein is increased significantly; and within a range of 35-40° C., the purities of both κ-casein and β-casein are decreased. Therefore, an equilibration temperature of 10-25° C. is preferred for the co-fractionation of κ- and β-caseins, and the most preferred temperature is 20° C.

TABLE 5
Co-fractionation results in equilibration temperature
regulation solutions in Example 3

| Equilibration | κ-casein | | β-casein | |
|---|---|---|---|---|
| temperature | Extraction | Purity | Extraction | Purity |
| (° C.) | rate (%) | (%) | rate (%) | (%) |
| 10 | 90.1 ± 0.3 | 16.2 ± 0.8 | 98.2 ± 1.1 | 64.1 ± 0.5 |
| 15 | 88.4 ± 0.8 | 16.6 ± 1.3 | 96.6 ± 1.3 | 64.3 ± 1.0 |
| 20 (Example 1) | 87.6 ± 1.0 | 17.8 ± 0.4 | 96.2 ± 0.7 | 74.1 ± 0.8 |
| 25 | 81.5 ± 0.8 | 18.0 ± 0.3 | 89.1 ± 0.8 | 74.1 ± 0.7 |
| 30 | 76.7 ± 1.1 | 18.9 ± 0.7 | 85.6 ± 1.0 | 74.2 ± 0.8 |
| 35 | 71.2 ± 0.7 | 19.5 ± 0.7 | 76.2 ± 0.9 | 74.5 ± 0.9 |
| 40 | 53.7 ± 0.4 | 18.8 ± 0.6 | 57.3 ± 0.7 | 71.8 ± 0.6 |

(4) Regulation of different equilibration durations: The stirring-assisted equilibration duration in the step (3) of Example 1 was adjusted to 0, 15, 30, 45, 75, and 90 minutes, respectively. All other steps remained consistent with those in Example 1.

In the equilibration duration regulation solutions, the extraction rates and purities of κ-casein and β-casein obtained in the step (6) are shown in Table 6. As can be seen from Table 6, the extraction rates of both κ-casein and β-casein are decreased gradually within a range of 0-75 minutes, and remain virtually unchanged within a range of 75-90 minutes. The purity of κ-casein is initially increased gradually over time, and stabilized after 60 minutes; and the purity of β-casein shows an increasing trend over time, with a significant increase observed within a range of 45-60 minutes, and becomes stabilized after 60 minutes. Therefore, an equilibration duration of 60-90 minutes is preferred for the co-fractionation of κ- and β-caseins.

TABLE 6
Co-fractionation results in equilibration
duration regulation solutions in Example 3

| Equilibration | κ-casein | | β-casein | |
|---|---|---|---|---|
| duration | Extraction | Purity | Extraction | Purity |
| (min) | rate (%) | (%) | rate (%) | (%) |
| 0 | 96.1 ± 0.7 | 14.2 ± 1.5 | 99.7 ± 0.7 | 57.8 ± 0.3 |
| 15 | 93.4 ± 0.6 | 14.8 ± 0.2 | 99.0 ± 1.1 | 61.2 ± 0.8 |
| 30 | 90.2 ± 0.4 | 15.9 ± 0.6 | 98.1 ± 1.3 | 66.8 ± 1.6 |
| 45 | 88.7 ± 1.1 | 16.5 ± 0.6 | 97.5 ± 0.7 | 69.5 ± 0.8 |
| 60 (Example 1) | 87.6 ± 1.0 | 17.8 ± 0.4 | 96.2 ± 0.7 | 74.1 ± 0.8 |
| 75 | 84.9 ± 0.9 | 17.9 ± 0.3 | 93.6 ± 0.8 | 75.7 ± 0.3 |
| 90 | 84.1 ± 1.2 | 18.2 ± 1.0 | 92.9 ± 0.5 | 76.2 ± 1.1 |

### Example 4: Co-Fractionation Option 1: Effects of Different Low-Temperature Treatment Temperatures and Low-Temperature Treatment Durations

(1) Regulation of different low-temperature treatment temperatures: The temperature in the step (4) of Example 1 was adjusted to 0, 2, 8, and 12° C., respectively. All other steps remained consistent with those in Example 1.

In the low-temperature treatment temperature regulation solutions, the extraction rates and purities of κ-casein and β-casein obtained in step (6) are shown in Table 7. As can be seen from Table 7, as the temperature is increased, the extraction rates of both κ-casein and β-casein show a decreasing trend. Specifically, the extraction rate of β-casein is decreased slightly within a range of 0-4° C., decreased significantly within a range of 4-8° C., and decreased slightly within a range of 8-12° C. As the temperature is increased, the purities of both κ-casein and β-casein show an increasing trend; and within a range of 4-12° C., the purity of β-casein remains virtually unchanged. Therefore, satisfactory results are achieved within the temperature range of 0-4° C. Specifically, a low-temperature treatment temperature of 4° C. is preferred for the co-fractionation of κ- and β-caseins.

TABLE 7
Co-fractionation results in low-temperature treatment
temperature regulation solutions in Example 4

| Low-temperature | | | | |
|---|---|---|---|---|
| treatment | κ-casein | | β-casein | |
| temperature | Extraction | Purity | Extraction | Purity |
| (° C.) | rate (%) | (%) | rate (%) | (%) |
| 0 | 89.2 ± 0.5 | 17.0 ± 0.6 | 98.7 ± 0.9 | 72.6 ± 1.0 |
| 2 | 87.9 ± 0.7 | 17.2 ± 0.2 | 96.2 ± 1.1 | 73.6 ± 0.2 |
| 4 (Example 1) | 87.6 ± 1.0 | 17.8 ± 0.4 | 96.2 ± 0.7 | 74.1 ± 0.8 |
| 8 | 85.5 ± 0.7 | 18.6 ± 0.7 | 91.8 ± 1.0 | 74.2 ± 0.8 |
| 12 | 84.8 ± 1.0 | 20.5 ± 0.9 | 89.0 ± 0.7 | 74.6 ± 0.9 |

(2) Regulation of different low-temperature treatment durations: A stirring treatment duration in the step (4) of Example 1 was adjusted to 0, 5, 20, 30, 40, 50, and 60 minutes, respectively. All other steps remained consistent with those in Example 1.

In the low-temperature treatment duration regulation solutions, the extraction rates and purities of κ-casein and β-casein obtained in step (6) are shown in Table 8. As can be seen from Table 8, the extraction rate of κ-casein is increased significantly within a range of 0-10 minutes and remains virtually unchanged within a range of 10-60 minutes; and the extraction rate of β-casein is increased significantly within a range of 0-10 minutes, remains virtually unchanged within a range of 10-50 minutes, and is decreased within a range of 50-60 minutes. The purity of κ-casein is increased significantly within a range of 0-10 minutes, is decreased slightly within a range of 10-20 minutes, and remains virtually unchanged within a range of 20-60 minutes; and the purity of β-casein is increased within a range the 0-10 minutes, remains virtually unchanged within a range of 10-50 minutes, and is increased slightly within a range of 50-60 minutes. Therefore, satisfactory results are achieved within the range of 10-50 minutes. Specifically, a low-temperature treatment duration of 10 minutes is preferred for the co-fractionation of κ- and β-caseins.

TABLE 8
Co-fractionation results in low-temperature treatment
duration regulation solutions in Example 4

| Low-temperature | κ-casein | | β-casein | |
|---|---|---|---|---|
| treatment | Extraction | Purity | Extraction | Purity |
| duration (min) | rate (%) | (%) | rate (%) | (%) |
| 0 | 84.2 ± 0.7 | 17.0 ± 1.5 | 91.3 ± 0.7 | 73.0 ± 0.3 |
| 5 | 85.6 ± 0.6 | 17.1 ± 0.2 | 93.3 ± 1.1 | 73.4 ± 0.8 |
| 10 (Example 1) | 87.6 ± 1.0 | 17.8 ± 0.4 | 96.2 ± 0.7 | 74.1 ± 0.8 |
| 20 | 87.5 ± 1.1 | 17.2 ± 0.6 | 96.1 ± 0.7 | 73.8 ± 1.2 |
| 30 | 87.6 ± 1.4 | 17.2 ± 0.4 | 96.1 ± 1.1 | 73.7 ± 0.2 |
| 40 | 87.6 ± 0.9 | 17.2 ± 0.3 | 96.2 ± 0.8 | 73.5 ± 0.3 |
| 50 | 87.6 ± 1.2 | 17.2 ± 1.0 | 96.3 ± 0.5 | 73.2 ± 1.1 |
| 60 | 87.5 ± 0.8 | 17.5 ± 0.4 | 94.5 ± 1.3 | 74.6 ± 0.5 |

### Example 5: Co-Fractionation Option 1: Effects of Different Acidifying Agents, Acid Precipitation pH Values, and Equilibration Durations

(1) Regulation of different acidifying agents: The acetic acid solution in the step (5) of Example 1 was adjusted to be a hydrochloric acid solution, a citric acid solution, or a lactic acid solution. All other steps remained consistent with those in Example 1.

In the acidifying agent regulation solutions, the extraction rates and purities of κ-casein and β-casein obtained in the step (6) are shown in Table 9. As can be seen from Table 9, an order of the extraction rates of both κ-casein and β-casein is: citric acid>lactic acid>acetic acid>hydrochloric acid; and an order of the purities of two caseins is: hydrochloric acid>acetic acid>lactic acid>citric acid. When the acetic acid was used as the acidifying agent, the purities of both κ-casein and β-casein are close to the purities achieved with hydrochloric acid, while the extraction rates of two caseins are higher than the extraction rates obtained with hydrochloric acid. Hydrochloric acid is a strong acid. Adding the high-concentration hydrochloric acid to the casein solution causes a significant local decrease in the pH, thereby leading to casein aggregation and a reduced extraction rate. Acetic acid is a weak acid. Adding the acetic acid enables more uniform acidification of the casein solution, thereby reducing casein aggregation caused by an excessively low local pH. Therefore, the acetic acid solution is the most preferred acidifying agent for the co-fractionation of κ- and β-caseins.

TABLE 9
Co-fractionation results in acidifying
agent regulation solutions in Example 5

| | κ-casein | | β-casein | |
|---|---|---|---|---|
| | Extraction | Purity | Extraction | Purity |
| Acidifying agent | rate (%) | (%) | rate (%) | (%) |
| Hydrochloric acid | 84.7 ± 0.9 | 17.9 ± 0.7 | 92.7 ± 0.4 | 74.4 ± 0.6 |
| Citric acid | 90.1 ± 0.4 | 16.8 ± 1.2 | 96.8 ± 1.0 | 70.2 ± 0.5 |
| Lactic acid | 88.9 ± 1.3 | 17.0 ± 0.9 | 96.3 ± 0.9 | 71.5 ± 0.6 |
| Acetic acid | 87.6 ± 1.0 | 17.8 ± 0.4 | 96.2 ± 0.7 | 74.1 ± 0.8 |
| (Example 1) | | | | |

(2) Regulation of different acid precipitation pH values: The acid precipitation pH in the step (5) of Example 1 was adjusted to 4.6, 4.7, 4.8, 4.9, 5.1, 5.2, 5.4, and 5.6, respectively. All other steps remained consistent with those in Example 1.

In the acid precipitation pH regulation solutions, the extraction rates and purities of κ-casein and β-casein obtained in the step (6) are shown in Table 10. As can be seen from Table 10, as the pH is increased, the extraction rates of both κ-casein and β-casein show a trend of first increasing and then decreasing. Specifically, the extraction rate of κ-casein reaches a maximum at pH 4.9, while the extraction rate of β-casein reaches a maximum at pH 5.0. Within a range of pH 4.6-4.9, the purities of both κ-casein and β-casein are increased significantly; within a range of pH 4.9-5.2, the purities of both κ-casein and β-casein are increased slightly; and within a range of pH 5.2-5.6, the purity of κ-casein is increased slightly, while the purity of β-casein is decreased. Therefore, satisfactory results are achieved within a range of pH 4.9-5.1. Specifically, an acid precipitation pH of 5.0 is preferred for the co-fractionation of κ- and β-caseins.

TABLE 10
Co-fractionation results in acid precipitation
pH regulation solutions in Example 5

| | κ-casein | | β-casein | |
|---|---|---|---|---|
| Acid | Extraction | Purity | Extraction | Purity |
| precipitation pH | rate (%) | (%) | rate (%) | (%) |
| 4.6 | 69.5 ± 0.7 | 15.3 ± 0.9 | 59.6 ± 0.6 | 63.9 ± 1.5 |
| 4.7 | 76.1 ± 1.4 | 16.2 ± 1.2 | 87.5 ± 1.1 | 69.6 ± 1.6 |
| 4.8 | 87.6 ± 1.1 | 17.2 ± 0.7 | 94.7 ± 1.4 | 70.5 ± 0.9 |
| 4.9 | 89.6 ± 0.9 | 17.5 ± 1.0 | 95.9 ± 1.2 | 72.6 ± 1.0 |
| 5.0 (Example 1) | 87.6 ± 1.0 | 17.8 ± 0.4 | 96.2 ± 0.7 | 74.1 ± 0.8 |
| 5.1 | 86.6 ± 1.0 | 17.9 ± 1.1 | 94.2 ± 0.6 | 74.1 ± 0.9 |
| 5.2 | 77.6 ± 0.6 | 18.2 ± 1.0 | 84.8 ± 1.2 | 74.2 ± 1.3 |
| 5.4 | 61.3 ± 0.7 | 18.4 ± 0.9 | 64.7 ± 0.9 | 73.7 ± 0.9 |
| 5.6 | 48.7 ± 1.1 | 19.3 ± 1.3 | 48.3 ± 0.4 | 72.4 ± 0.6 |

(3) Regulation of different equilibration durations: The stirring-assisted equilibration duration in the step (5) of Example 1 was adjusted to 0, 1, 2, 4, 12, and 24 hours, respectively. All other steps remained consistent with those in Example 1.

In the equilibration duration regulation solutions, the extraction rates and purities of κ-casein and β-casein obtained in the step (6) are shown in Table 11. As can be seen from Table 11, the extraction rates of both κ-casein and β-casein are increased significantly within a range of 0-8 hours, and remain virtually unchanged within a range of 8-24 hours. The purity of κ-casein is gradually decreased within a range of 0-4 hours, is increased within a range of 4-8 hours, and remains virtually unchanged within a range of 8-24 hours; and the purity of β-casein is gradually decreased over an entire period of 0-24 hours. Therefore, satisfactory extraction rates and purities are achieved within the range of 8-24 hours; and an equilibration duration of 8 hours is preferred for the co-fractionation of κ- and β-caseins.

TABLE 11
Co-fractionation results in equilibration
duration regulation solutions in Example 5

| Equilibration | κ-casein | | β-casein | |
|---|---|---|---|---|
| duration | Extraction | Purity | Extraction | Purity |
| (h) | rate (%) | (%) | rate (%) | (%) |
| 0 | 70.6 ± 1.7 | 17.8 ± 0.6 | 76.0 ± 0.8 | 75.6 ± 1.3 |
| 1 | 77.5 ± 1.2 | 17.4 ± 1.3 | 85.1 ± 0.5 | 75.6 ± 1.1 |
| 2 | 80.9 ± 0.8 | 16.9 ± 0.5 | 90.6 ± 1.3 | 75.6 ± 0.9 |
| 4 | 83.3 ± 0.6 | 16.9 ± 0.7 | 93.2 ± 1.0 | 74.7 ± 1.4 |
| 8 (Example 1) | 87.6 ± 1.0 | 17.8 ± 0.4 | 96.2 ± 0.7 | 74.1 ± 0.8 |
| 12 | 88.4 ± 0.4 | 17.6 ± 0.6 | 96.3 ± 1.3 | 73.2 ± 1.6 |
| 24 | 88.6 ± 1.4 | 17.8 ± 0.6 | 96.4 ± 1.1 | 73.0 ± 1.5 |

### Example 6: Co-Fractionation Option 1: Effects of Different Precipitation pH Values, Precipitation Temperatures, and Precipitation Durations

(1) Regulation of different precipitation pH values: The pH after the addition of the hydrochloric acid solution in the step (7) of Example 1 was adjusted to 3.0, 3.2, 3.4, 3.6, 3.8, 4.0, 4.2, 4.4, 4.6, 4.8, 5.0, and 5.2, respectively, and the subsequent equilibration treatment was adjusted to stirring at 25° C. for 30 minutes. All other steps remained consistent with those in Example 1.

In the precipitation pH regulation solutions, the precipitation rates of κ-casein and β-casein obtained in the step (7) are shown in Table 12. As can be seen from Table 12, the precipitation rates of both κ-casein and β-casein show an increasing trend within a range of pH 3.0-3.6, and a decreasing trend within a range of pH 3.6-5.2. Therefore, a pH of 3.4-3.8 is preferred for the precipitation and recovery of κ- and β-caseins, and the most preferred pH is 3.6.

TABLE 12
Co-fractionation results in precipitation
pH regulation solutions in Example 6

| | Precipitation rate of | Precipitation rate of |
|---|---|---|
| Precipitation pH | κ-casein (%) | β-casein (%) |
| 3.0 | 71.3 ± 0.4 | 96.2 ± 0.5 |
| 3.2 | 74.3 ± 0.2 | 97.3 ± 0.8 |
| 3.4 | 78.5 ± 1.8 | 97.9 ± 0.8 |
| 3.6 | 83.8 ± 0.1 | 98.1 ± 0.1 |
| 3.8 | 81.5 ± 0.7 | 97.9 ± 0.2 |
| 4.0 | 80.9 ± 0.4 | 96.8 ± 0.6 |
| 4.2 | 77.9 ± 0.6 | 94.8 ± 1.6 |
| 4.4 | 69.5 ± 0.6 | 92.2 ± 1.0 |
| 4.6 | 50.6 ± 0.2 | 78.6 ± 0.8 |
| 4.8 | 33.4 ± 2.0 | 57.5 ± 0.2 |
| 5.0 | 27.6 ± 1.2 | 24.3 ± 1.8 |
| 5.2 | 14.4 ± 0.5 | 19.1 ± 0.4 |

(2) Regulation of different precipitation temperatures: The equilibration treatment in the step (7) of Example 1 was adjusted to stirring at 15, 25, 35, 45, 50, and 55° C. for 30 minutes, respectively. All other steps remained consistent with those in Example 1.

In the precipitation temperature regulation solutions, the precipitation rates of κ-casein and β-casein obtained in the step (7) are shown in Table 13. As can be seen from Table 13, the precipitation rate of κ-casein is increased gradually within a range of 15-50° C., and remains virtually unchanged within a range of 50-55° C. The extraction rate of β-casein is increased gradually within a range of 15-45° C., and remains virtually unchanged within a range of 45-55° C. Therefore, a temperature of 45-55° C. is preferred for the precipitation and recovery of κ- and β-caseins, and the most preferred temperature is 50° C.

TABLE 1
Co-fractionation results in precipitation temperature
regulation solutions in Example 6

| Precipitation | Precipitation rate of | Precipitation rate of |
|---|---|---|
| temperature (° C.) | κ-casein (%) | β-casein (%) |
| 15 | 75.5 ± 2.1 | 93.0 ± 0.8 |
| 25 | 83.8 ± 0.1 | 98.1 ± 0.1 |
| 35 | 87.6 ± 0.8 | 98.4 ± 0.3 |
| 45 | 90.1 ± 2.1 | 99.2 ± 0.1 |
| 50 | 93.5 ± 0.6 | 99.9 ± 0.2 |
| 55 | 93.8 ± 0.2 | 99.9 ± 0.6 |

(3) Regulation of different precipitation durations: The precipitation duration in the step (7) of Example 1 was adjusted to 0, 10, 30, 40, 50, and 60 minutes, respectively. All other steps remained consistent with those in Example 1.

In the precipitation duration regulation solutions, the precipitation rates of κ-casein and β-casein obtained in the step (7) are shown in Table 14. As can be seen from Table 14, the precipitation rate of κ-casein is increased significantly within a range of 0-20 minutes, and decreased slightly within a range of 20-60 minutes. The precipitation rate of β-casein remains unchanged over time. Therefore, a precipitation duration of 20-60 minutes is preferred for the precipitation and recovery of κ- and β-caseins, and the most preferred duration is 20 minutes.

TABLE 2
Co-fractionation results in precipitation
duration regulation solutions in Example 6

| | Precipitation rate of | Precipitation rate of |
|---|---|---|
| Precipitation duration (min) | κ-casein (%) | β-casein (%) |
| 0 | 87.6 ± 1.6 | 99.9 ± 0.1 |
| 10 | 90.2 ± 2.0 | 99.9 ± 0.1 |
| 20 (Example 1) | 93.7 ± 0.5 | 99.9 ± 0.1 |
| 30 | 93.5 ± 0.6 | 99.9 ± 0.2 |
| 40 | 93.4 ± 0.1 | 99.9 ± 0.1 |
| 50 | 93.4 ± 0.3 | 99.9 ± 0.1 |
| 60 | 93.3 ± 0.2 | 99.9 ± 0.1 |

### Example 7: Co-Fractionation Option 2 for κ- and β-Caseins in MCC

A method for co-fractionation of κ- and β-caseins includes the following steps:

(1) MCC powder was taken and reconstituted in ultrapure water to obtain a reconstituted casein solution having a casein mass concentration of 3%.

(2) At 25° C., a 2 M HCl solution was added to the reconstituted casein solution to adjust a pH of the reconstituted casein solution to 4.3, and a resulting solution was equilibrated under stirring (300 rpm) for 30 minutes after being heated to 45° C., and centrifuged at 5,000 g for 10 min after being cooled to 25° C. to obtain a precipitate.

(3) The precipitate was collected and placed in ultrapure water, a 2 M NaOH solution was added to adjust a pH to 7.5 at 25° C., and a resulting solution was equilibrated under stirring (300 rpm) at 25° C. for 60 minutes to obtain a reconstituted casein solution having a casein mass concentration of 3%.

(4) A 2 M calcium chloride solution was added until a concentration of added calcium ions reached 45 mM, and a resulting solution was equilibrated under stirring (300 rpm) at 25° C. for 60 minutes to obtain a casein-calcium complexed solution.

(5) A 4 M acetic acid solution was added to adjust a pH of the complexed solution to 5.0 for acid precipitation, and a resulting solution was equilibrated under stirring (300 rpm) at 25° C. for 90 minutes.

(6) The casein-calcium complexed solution was subjected to low-temperature equilibration under stirring (300 rpm) for 8 hours after being cooled to 2° C.

(7) Centrifugation was performed at 2° C. and 3,000 g for 10 minutes to obtain a precipitate enriched with αₛ-casein and a supernatant enriched with κ- and β-caseins.

(8) The supernatant enriched with κ- and β-caseins was collected, a 2 M hydrochloric acid solution was added at 25° C. to adjust a pH of the supernatant to 3.6, and a resulting solution was equilibrated under stirring (300 rpm) for 20 minutes after being heated to 50° C., and centrifuged at 10,000 g for 10 minutes after being cooled to 25° C. to obtain a precipitate enriched with κ- and β-caseins and a supernatant depleted in κ- and β-caseins.

The yields and purities of κ-casein and β-casein obtained in step (8) are shown in Table 15. As can be seen from Table 15, the method of Example 7 is capable of effectively co-fractionating κ- and β-caseins, and the yields and purities of κ- and β-caseins are high.

TABLE 15
Co-fractionation results of κ- and β-caseins in Example 7

| Casein component | Yield (%) | Purity (%) |
|---|---|---|
| κ-casein | 73.5 ± 0.4 | 15.2 ± 0.3 |
| β-casein | 97.7 ± 0.5 | 67.7 ± 0.3 |

### Example 8: Co-Fractionation Option 2: Effects of Different Reconstitution pH Values, and Equilibration Durations

(1) Regulation of different reconstitution pH values: The pH in the step (3) of Example 7 was adjusted to 6.5, 7.0, 8.0, 8.5, and 9.0, respectively. All other steps remained consistent with those in Example 7.

In the reconstitution pH regulation solutions, the extraction rates and purities of κ-casein and β-casein obtained in the step (7) are shown in Table 16. As can be seen from Table 16, within a range of pH 6.5-7.5, the extraction rates of both κ-casein and β-casein are increased significantly; and the purity of κ-casein shows a decreasing trend, while the purity of β-casein shows an increasing trend. Within a range of pH 7.5-9.0, the extraction rates of both κ-casein and β-casein are gradually decreased; and the purity of κ-casein is increased slightly, and the purity of β-casein is decreased significantly. At a low pH, incomplete casein reconstitution results in a lower extraction rate. As the pH is increased, net negative charges of the caseins are gradually increased, such that a calcium-binding degree of the caseins is enhanced, thereby leading to a decrease in the extraction rate. Therefore, a reconstitution pH of 7.5 is preferred.

TABLE 16
Co-fractionation results in reconstitution
pH regulation solutions in Example 8

| | κ-casein | | β-casein | |
|---|---|---|---|---|
| Reconstitution | Extraction | Purity | Extraction | Purity |
| pH | rate (%) | (%) | rate (%) | (%) |
| 6.5 | 78.8 ± 0.7 | 18.6 ± 0.9 | 77.3 ± 0.6 | 64.7 ± 1.5 |
| 7.0 | 79.9 ± 1.4 | 18.5 ± 1.2 | 82.5 ± 1.1 | 64.7 ± 1.6 |
| 7.5 (Example 7) | 84.5 ± 0.5 | 17.7 ± 0.2 | 97.8 ± 0.6 | 68.1 ± 0.2 |
| 8.0 | 80.6 ± 0.9 | 18.5 ± 1.0 | 83.9 ± 1.2 | 65.8 ± 1.0 |
| 8.5 | 80.7 ± 1.0 | 20.9 ± 0.4 | 70.3 ± 0.7 | 62.0 ± 0.8 |
| 9.0 | 74.5 ± 0.5 | 20.7 ± 1.1 | 64.5 ± 0.6 | 61.4 ± 0.9 |

(2) Regulation of different equilibration durations: The equilibration duration in the step (3) of Example 7 was adjusted to 0, 15, 30, 45, and 75 minutes, respectively. All other steps remained consistent with those in Example 7.

In the equilibration duration regulation solutions, the extraction rates and purities of κ-casein and β-casein obtained in the step (7) are shown in Table 17. As can be seen from Table 17, the extraction rates of both κ-casein and β-casein are decreased gradually within a range of 0-60 minutes, and remain virtually unchanged within a range of 60-75 minutes, indicating that prolonging the duration promotes the reconstitution of acid-precipitated caseins, thereby improving the extraction rates of the caseins. Within a range of 0-60 minutes, the purity of κ-casein shows a decreasing trend, while the purity of β-casein shows an increasing trend; and within a range of 60-75 minutes, the purities of both κ-casein and β-casein remain virtually unchanged. Therefore, satisfactory results are achieved within the range of 60-75 minutes. Specifically, an equilibration duration of 60 minutes is preferred for the co-fractionation of κ- and β-caseins.

TABLE 17
Co-fractionation results in equilibration
duration regulation solutions in Example 8

| Equilibration | κ-casein | | β-casein | |
|---|---|---|---|---|
| duration | Extraction | Purity | Extraction | Purity |
| (min) | rate (%) | (%) | rate (%) | (%) |
| 0 | 71.9 ± 0.7 | 19.5 ± 1.5 | 69.8 ± 0.7 | 62.6 ± 0.3 |
| 15 | 74.0 ± 0.6 | 19.1 ± 0.2 | 73.4 ± 1.1 | 63.1 ± 0.8 |
| 30 | 77.5 ± 0.4 | 19.0 ± 0.6 | 79.2 ± 1.3 | 64.6 ± 1.6 |
| 45 | 84.7 ± 1.1 | 18.8 ± 0.6 | 89.7 ± 0.7 | 66.2 ± 0.8 |
| 60 (Example 7) | 84.5 ± 0.5 | 17.7 ± 0.2 | 97.8 ± 0.6 | 68.1 ± 0.2 |
| 75 | 85.7 ± 1.2 | 17.9 ± 1.0 | 98.0 ± 0.5 | 67.9 ± 1.1 |

### Example 9: Co-Fractionation Option 2: Effect of Different Low-Temperature Starting Positions

Control of different low-temperature starting positions:

(1) After reconstitution:

The low-temperature treatment in the step (6) of Example 7 was adjusted to follow the step (3). Specifically, the reconstituted casein solution was cooled to 2° C., and the temperature in the subsequent steps was adjusted to 2° C. All other steps remained consistent with those in Example 7.

(2) After casein-calcium complexation:

The low-temperature treatment in the step (6) of Example 7 was adjusted to follow the step (4). Specifically, the casein-calcium complexed solution was cooled to 2° C., and the temperature in the subsequent steps was adjusted to 2° C. All other steps remained consistent with those in Example 7.

(3) After αₛ-casein precipitation:

All other steps remained consistent with those in Example 7.

In the low-temperature starting position regulation solutions, the extraction rates and purities of κ-casein and β-casein obtained in step (7) are shown in Table 18. As can be seen from Table 18, as the low-temperature starting position is delayed in the process, the extraction rate of κ-casein shows a decreasing trend. Specifically, when the low-temperature step starting position occurs after the αₛ-casein precipitation, the purity of β-casein is increased significantly. Delaying the low-temperature step starting position enhances the calcium-binding capacity of the caseins, thereby strengthening the selective precipitation of the caseins by calcium ions and resulting in increased purity of the co-fractionated β-casein. Therefore, the low-temperature starting position occurring after the αₛ-casein precipitation is preferred for the co-fractionation of κ- and β-caseins.

TABLE 18
Co-fractionation results in low-temperature starting
position regulation solutions in Example 9

| | κ-casein | | β-casein | |
|---|---|---|---|---|
| Low-temperature | Extraction | Purity | Extraction | Purity |
| starting position | rate (%) | (%) | rate (%) | (%) |
| After reconstitution | 95.5 ± 0.6 | 17.9 ± 0.2 | 97.7 ± 1.6 | 50.6 ± 0.7 |
| After casein-calcium | 94.5 ± 0.7 | 17.6 ± 0.4 | 97.8 ± 0.7 | 50.9 ± 0.5 |
| complexation | | | | |
| After αₛ-casein | 84.5 ± 0.4 | 17.7 ± 0.6 | 97.8 ± 0.9 | 68.1 ± 0.5 |
| precipitation | | | | |
| (Example 7) | | | | |

### Example 10: Co-Fractionation Option 2: Effects of Different Concentrations of Added Calcium Ions, and Equilibration Temperatures

(1) Regulation of different concentrations of added calcium ions: The concentration of added calcium ions in the step (4) of Example 7 was adjusted to 30, 35, 40, 50, 55, 60, 65, and 70 mM, respectively. All other steps remained consistent with those in Example 7.

In the regulation solutions for the concentration of added calcium ions, the extraction rates and purities of κ-casein and β-casein obtained in the step (7) are shown in Table 19. As can be seen from Table 19, when the concentration of added calcium ions falls within a range of 30-45 mM range, the extraction rates of both κ-casein and β-casein show an increasing trend, while the purity of κ-casein shows an increasing trend and the purity of β-casein shows a decreasing trend. When the concentration of added calcium ions falls within a range of 45-70 mM, the extraction rate of κ-casein is further increased, the extraction rate of β-casein remains virtually unchanged, the purity of κ-casein is further increased, and the purity of β-casein is decreased significantly. Therefore, a concentration of added calcium ions of 45 mM is preferred for the co-fractionation of κ- and β-caseins.

TABLE 19
Co-fractionation results in regulation solutions for
the concentration of added calcium ions in Example 10

| Calcium ion | κ-casein | | β-casein | |
|---|---|---|---|---|
| concentration | Extraction | Purity | Extraction | Purity |
| (mM) | rate (%) | (%) | rate (%) | (%) |
| 30 | 56.7 ± 1.1 | 13.8 ± 0.4 | 83.1 ± 0.5 | 76.1 ± 0.5 |
| 35 | 65.2 ± 0.8 | 15.8 ± 0.6 | 83.4 ± 1.0 | 74.2 ± 1.1 |
| 40 | 72.9 ± 1.0 | 16.7 ± 0.7 | 92.9 ± 0.9 | 70.4 ± 0.9 |
| 45 (Example 7) | 84.5 ± 0.5 | 17.7 ± 0.2 | 97.8 ± 0.6 | 68.1 ± 0.2 |
| 50 | 91.0 ± 0.4 | 19.1 ± 0.6 | 98.1 ± 0.9 | 62.9 ± 0.5 |
| 55 | 94.3 ± 0.5 | 20.4 ± 0.6 | 98.4 ± 0.9 | 61.3 ± 0.7 |
| 60 | 94.5 ± 0.5 | 21.2 ± 0.2 | 98.8 ± 0.6 | 59.1 ± 0.2 |
| 65 | 96.1 ± 0.6 | 21.9 ± 0.9 | 98.2 ± 1.1 | 58.4 ± 0.8 |
| 70 | 96.8 ± 1.0 | 22.5 ± 0.5 | 98.9 ± 0.8 | 57.1 ± 0.8 |

(2) Regulation of different equilibration temperatures: The equilibration temperature in the step (4) of Example 7 was adjusted to 10, 15, 20, 30, 35, and 40° C., respectively. All other steps remained consistent with those in Example 7.

In the equilibration temperature regulation solutions, the extraction rates and purities of κ-casein and β-casein obtained in the step (7) are shown in Table 20. As can be seen from Table 20, as the temperature is increased, the extraction rates of both κ-casein and β-casein show a decreasing trend, while the purities of both κ-casein and β-casein are first increased and then decreased, and reach maximums at 25° C. Therefore, an equilibration temperature of 25° C. is preferred for the co-fractionation of κ- and β-caseins.

TABLE 20
Co-fractionation results in equilibration temperature
regulation solutions in Example 10

| Calcium | | | | |
|---|---|---|---|---|
| equilibration | κ-casein | | β-casein | |
| temperature | Extraction | Purity | Extraction | Purity |
| (° C.) | rate (%) | (%) | rate (%) | (%) |
| 10 | 90.5 ± 0.5 | 15.3 ± 0.2 | 98.8 ± 0.6 | 64.1 ± 0.2 |
| 15 | 89.3 ± 0.5 | 15.8 ± 0.7 | 98.7 ± 0.9 | 65.3 ± 0.7 |
| 20 | 87.5 ± 1.0 | 16.1 ± 0.7 | 98.1 ± 0.9 | 66.9 ± 0.9 |
| 25 (Example 7) | 84.5 ± 0.5 | 17.7 ± 0.2 | 97.8 ± 0.6 | 68.1 ± 0.2 |
| 30 | 84.1 ± 0.2 | 17.4 ± 0.6 | 96.1 ± 0.9 | 67.5 ± 0.5 |
| 35 | 81.2 ± 0.8 | 17.3 ± 0.6 | 95.4 ± 1.0 | 66.7 ± 1.1 |
| 40 | 80.7 ± 1.3 | 17.0 ± 0.4 | 95.1 ± 0.5 | 66.6 ± 0.5 |

### Example 11: Co-Fractionation Option 2: Effects of Different Acid Precipitation Equilibration Durations, and Low-Temperature Treatment Temperatures

(1) Regulation of different acid precipitation equilibration durations: The stirring-assisted equilibration duration in the step (5) of Example 7 was adjusted to 0, 30, 60, 120, and 150 minutes, respectively. All other steps remained consistent with those in Example 7.

In the acid precipitation equilibration duration regulation solutions, the extraction rates and purities of κ-casein and β-casein obtained in the step (7) are shown in Table 21. As can be seen from Table 21, within a range of 0-90 minutes, the extraction rates and purities of both κ-casein and β-casein are gradually increased; and within a range of 90-150 minutes, the extraction rates and purities of both κ-casein and β-casein show no significant change. Therefore, high extraction rates and purities are achieved within the range of 90-150 minutes. Specifically, an equilibration duration of 90 minutes is preferred for the co-fractionation of κ- and β-caseins.

TABLE 21
Co-fractionation results in acid precipitation equilibration
duration regulation solutions in Example 11

| Equilibration | κ-casein | | β-casein | |
|---|---|---|---|---|
| duration | Extraction | Purity | Extraction | Purity |
| (min) | rate (%) | (%) | rate (%) | (%) |
| 0 | 81.4 ± 1.0 | 8.9 ± 1.4 | 94.9 ± 0.7 | 57.1 ± 0.8 |
| 30 | 82.4 ± 0.6 | 11.8 ± 0.2 | 96.4 ± 1.1 | 59.2 ± 0.8 |
| 60 | 83.5 ± 0.5 | 16.7 ± 0.2 | 97.1 ± 0.6 | 65.1 ± 0.2 |
| 90 (Example 7) | 84.5 ± 0.5 | 17.7 ± 0.2 | 97.8 ± 0.6 | 68.1 ± 0.2 |
| 120 | 84.7 ± 1.0 | 17.9 ± 0.4 | 98.3 ± 0.7 | 68.3 ± 0.8 |
| 150 | 84.1 ± 1.2 | 17.8 ± 1.0 | 98.3 ± 0.5 | 67.8 ± 1.1 |

(2) Regulation of different low-temperature treatment temperatures: The temperature in the step (6) of Example 7 was adjusted to 0, 4, 8, 12, and 24° C., respectively. All other steps remained consistent with those in Example 7.

In the low-temperature treatment temperature regulation solutions, the extraction rates and purities of κ-casein and β-casein obtained in step (7) are shown in Table 22. As can be seen from Table 22, as the temperature is increased, the extraction rates of both κ-casein and β-casein show a decreasing trend. Specifically, the extraction rate of β-casein is decreased slightly within a range of 0-2° C., and decreased significantly within a range of 2-12° C. As the temperature is increased, the purities of both κ-casein and β-casein are increased slightly. Therefore, satisfactory results are achieved within the temperature range of 0-2° C. Specifically, a low-temperature treatment temperature of 2° C. is preferred for the co-fractionation of κ- and β-caseins.

TABLE 22
Co-fractionation results in low-temperature treatment
temperature regulation solutions in Example 11

| Low-temperature | | | | |
|---|---|---|---|---|
| treatment | κ-casein | | β-casein | |
| temperature | Extraction | Purity | Extraction | Purity |
| (° C.) | rate (%) | (%) | rate (%) | (%) |
| 0 | 86.2 ± 0.5 | 17.6 ± 0.6 | 98.4 ± 0.6 | 68.6 ± 1.0 |
| 2 (Example 7) | 84.5 ± 0.5 | 17.7 ± 0.2 | 97.8 ± 0.6 | 68.1 ± 0.2 |
| 4 | 82.5 ± 1.0 | 18.2 ± 0.4 | 93.3 ± 0.9 | 71.2 ± 0.7 |
| 8 | 81.9 ± 0.7 | 19.3 ± 0.7 | 93.2 ± 1.0 | 72.3 ± 0.5 |
| 12 | 81.8 ± 1.0 | 21.1 ± 0.9 | 90.1 ± 0.7 | 73.6 ± 0.9 |

### Example 12: Further Optimization of Co-Fractionation Option 2 for κ- and β-Caseins in MCC: Effect of Different Acid Precipitation Equilibration Temperatures

The temperature in the step (5) of Example 7 was adjusted to 19, 21, 23, 27, 29, and 31° C., respectively. All other steps remained consistent with those in Example 7.

In the acid precipitation equilibration temperature regulation solutions, the extraction rates and purities of κ-casein and β-casein obtained in the step (7) are shown in Table 23. As can be seen from Table 23, as the temperature is increased, the extraction rates and purities of both κ-casein and β-casein show a trend of first increasing and then decreasing, are decreased significantly at 29° C., and reach relatively high values within a range of 23-27° C. Therefore, an acid precipitation equilibration temperature of 23-27° C. is preferred for the co-fractionation of κ- and β-caseins, and the most preferred acid precipitation equilibration temperature is 27° C.

TABLE 23
Co-fractionation results in acid precipitation equilibration
temperature regulation solutions in Example 12

| Equilibration | κ-casein | | β-casein | |
|---|---|---|---|---|
| temperature | Extraction | Purity | Extraction | Purity |
| (° C.) | rate (%) | (%) | rate (%) | (%) |
| 19 | 83.8 ± 1.1 | 15.9 ± 1.4 | 96.9 ± 0.7 | 64.1 ± 0.8 |
| 21 | 84.0 ± 0.6 | 16.6 ± 0.2 | 97.0 ± 1.1 | 65.2 ± 0.8 |
| 23 | 84.1 ± 0.5 | 17.1 ± 0.2 | 97.1 ± 0.6 | 67.1 ± 0.2 |
| 25 (Example 7) | 84.5 ± 0.5 | 17.7 ± 0.2 | 97.8 ± 0.6 | 68.1 ± 0.2 |
| 27 | 84.9 ± 1.0 | 18.2 ± 0.4 | 98.1 ± 0.7 | 69.1 ± 0.8 |
| 29 | 82.1 ± 1.0 | 17.8 ± 0.7 | 95.3 ± 0.4 | 67.8 ± 1.0 |
| 31 | 81.8 ± 1.2 | 17.6 ± 1.0 | 95.0 ± 0.5 | 67.5 ± 1.1 |

### Example 13: Further Optimization of Co-Fractionation Option 2 for κ- and β-Caseins in MCC: Effect of Different Low-Temperature Equilibration Durations

The duration in the step (6) of Example 12 was adjusted to 5, 6, 7, 9, 10, and 11 hours, respectively, and the temperature in the step (5) was adjusted to 27° C. All other steps remained consistent with those in Example 12.

In the low-temperature equilibration duration regulation solutions, the extraction rates and purities of κ-casein and β-casein obtained in the step (7) are shown in Table 24. As can be seen from Table 24, the extraction rates and purities of both κ-casein and β-casein show an increasing trend over time. Specifically, the extraction rates and purities of both κ-casein and β-casein are increased significantly within a range of 5-7 hours, are increased slightly within a range of 7-9 hours, and remain unchanged within a range of 9-11 hours. Therefore, a low-temperature equilibration duration of 7-9 hours is preferred for the co-fractionation of κ- and β-caseins, and the most preferred duration is 9 hours.

TABLE 24
Co-fractionation results in low-temperature equilibration
duration regulation solutions in Example 13

| Low-temperature | κ-casein | | β-casein | |
|---|---|---|---|---|
| duration | Extraction | Purity | Extraction | Purity |
| (h) | rate (%) | (%) | rate (%) | (%) |
| 5 | 80.4 ± 1.1 | 16.9 ± 1.4 | 93.9 ± 0.7 | 65.1 ± 0.8 |
| 6 | 82.1 ± 0.6 | 17.1 ± 0.2 | 95.4 ± 1.1 | 66.2 ± 0.8 |
| 7 | 84.7 ± 0.5 | 17.9 ± 0.2 | 97.9 ± 0.6 | 68.9 ± 0.2 |
| 8 (Example 12) | 84.9 ± 1.0 | 18.2 ± 0.4 | 98.1 ± 0.7 | 69.1 ± 0.8 |
| 9 | 86.1 ± 1.0 | 18.5 ± 0.8 | 98.9 ± 0.9 | 69.3 ± 0.8 |
| 10 | 86.0 ± 0.7 | 18.4 ± 0.4 | 98.8 ± 0.6 | 69.2 ± 0.8 |
| 11 | 86.0 ± 0.6 | 18.3 ± 0.3 | 98.6 ± 0.5 | 69.1 ± 0.7 |

### Example 14: Further Optimization of Co-Fractionation Option 2 for κ- and β-Caseins in MCC: Effect of Different Casein Mass Concentrations

The casein mass concentration in the step (1) of Example 13 was adjusted to 2, 4, 5, 6, and 7%, respectively, and the duration in the step (6) was adjusted to 9 hours. All other steps remained consistent with those in Example 13.

In the casein mass concentration regulation solutions, the extraction rates and purities of κ-casein and β-casein obtained in the step (7) are shown in Table 25. As can be seen from Table 25, as the casein mass concentration is increased, the extraction rates and purities of both κ-casein and β-casein show a trend of first increasing and then decreasing, and reach relatively high values within a range of 2-4%. Therefore, a casein mass concentration of 2-4% is preferred for the co-fractionation of κ- and β-caseins, and the most preferred casein mass concentration is 4%.

TABLE 25
Co-fractionation results in casein mass concentration
regulation solutions in Example 14

| Casein mass | κ-casein | | β-casein | |
|---|---|---|---|---|
| concentration | Extraction | Purity | Extraction | Purity |
| (%) | rate (%) | (%) | rate (%) | (%) |
| 2 | 84.4 ± 1.1 | 18.1 ± 1.4 | 97.9 ± 0.7 | 68.1 ± 0.8 |
| 3 (Example 13) | 86.1 ± 1.0 | 18.5 ± 0.8 | 98.9 ± 0.9 | 69.3 ± 0.8 |
| 4 | 89.7 ± 0.5 | 19.4 ± 0.2 | 99.4 ± 0.6 | 69.9 ± 0.2 |
| 5 | 84.0 ± 0.2 | 17.8 ± 0.6 | 96.6 ± 0.9 | 67.3 ± 0.5 |
| 6 | 83.1 ± 1.0 | 17.0 ± 0.4 | 95.9 ± 0.7 | 63.1 ± 0.8 |
| 7 | 82.1 ± 0.2 | 16.4 ± 0.2 | 94.8 ± 0.5 | 60.0 ± 0.5 |

### Comparative Example 3: Extraction of κ- and β-Caseins Under Strong Alkaline Conditions

Extraction of κ- and β-caseins under strong alkaline conditions includes the following steps:

The casein mass concentration in step (1) of Example 14 was selected as 4%, and the pH in step (3) of Example 14 was adjusted to 11. All other steps remained consistent with those in Example 14.

The yields and purities of κ-casein and β-casein obtained in the step (8) in Example 14 and Comparative Example 3 are shown in Table 26. As can be seen from Table 26, compared with the strong alkaline process of Comparative Example 3, the weak alkaline process of Example 14 achieves significantly higher yields and purities of both κ-casein and β-casein. Furthermore, the purities of both κ-casein and β-casein are closer to the purities of both κ-casein and β-casein in human milk, and a content of αₛ₂-casein is extremely low, indicating that the casein composition obtained by weak alkaline co-extraction is closer to the casein composition of the human milk. Moreover, compared with the strong alkaline process, the extent of dephosphorylation and deamidation reactions occurring in the weak alkaline process is minimal, and a content of a harmful substance lysinoalanine generated is also extremely low, indicating the mildness and safety of the weak alkaline process.

TABLE 26
Co-fractionation results of κ- and β-caseins in Example 14 and Comparative Example 3

| | | | | Phosphate group | Amide group | |
|---|---|---|---|---|---|---|
| | | | | removal amount | removal amount | Lysinoalanine |
| | | Yield | Purity | (μg phosphorus/g | (μg ammonia/g | content (μg/g |
| Example | Casein | (%) | (%) | protein) | protein) | protein) |
| Example 14 | κ-casein | 85.7 ± 0.5 | 18.3 ± 0.2 | 0.10 | 1.5 | 0.77 |
| (4% casein) | αₛ₁-casein | 20.5 ± 0.9 | 9.9 ± 0.8 | | | |
| | αₛ₂-casein | 4.6 ± 0.7 | 2.2 ± 1.1 | | | |
| | β-casein | 98.7 ± 0.6 | 69.6 ± 0.2 | | | |
| Comparative | κ-casein | 70.5 ± 0.5 | 17.6 ± 1.1 | 25.7 | 58.2 | 193.7 |
| Example 3 | αₛ₁-casein | 22.4 ± 1.2 | 11.1 ± 0.8 | | | |
| (4% casein) | αₛ₂-casein | 19.4 ± 0.7 | 10.6 ± 1.1 | | | |
| | β-casein | 64.1 ± 0.6 | 60.7 ± 0.9 | | | |

### Example 15: Further Optimization of Co-Fractionation Option 2 for κ- and β-Caseins in MCC: Effect of Different Concentrations of Added Calcium Ions

The concentration of added calcium ions in the step (4) of Example 14 was adjusted to 40, 50, 55, 60, 65, 70, 75, and 80 mM, respectively, and the casein mass concentration in the step (1) was adjusted to 4%. All other steps remained consistent with those in Example 14.

In the regulation solutions for the concentration of added calcium ions, the yields and purities of κ-casein and β-casein obtained in the step (8) are shown in Table 27. As can be seen from Table 27, when the concentration of added calcium ions falls within a range of 40-65 mM, the yields and purities of both κ-casein and β-casein show an increasing trend, with κ-casein increased more significantly, while the yields and purities of both αₛ₁-casein and αₛ₂-casein show a decreasing trend; when the concentration of added calcium ions falls within a range of 65-70 mM, the yields and purities of individual caseins remain unchanged, the resulting co-fractionated product contains no αₛ₂-casein, and the purities of κ-casein and β-casein are similar to the purities of κ-casein and β-casein in human milk, indicating that the casein composition of the co-fractionated product is similar to the casein composition of the human milk; when the concentration of added calcium ions falls within a range of 70-80 mM, the purity of κ-casein is increased significantly, while both the yield and purity of β-casein are decreased significantly. Therefore, a concentration of added calcium ions of 45-70 mM is preferred for the co-fractionation of κ- and β-caseins, and the most preferred concentration of added calcium ions is 65-70 mM.

TABLE 27
Co-fractionation results in calcium ion concentration regulation solutions in Example 15

| Calcium ion | | | | | | | | |
|---|---|---|---|---|---|---|---|---|
| concentration | κ-casein | | αₛ₁-casein | | αₛ₂-casein | | β-casein | |
| (mM) | Yield (%) | Purity (%) | Yield (%) | Purity (%) | Yield (%) | Purity (%) | Yield (%) | Purity (%) |
| 40 | 82.9 ± 0.4 | 16.9 ± 0.3 | 21.6 ± 0.8 | 10.4 ± 0.7 | 5.8 ± 0.8 | 3.5 ± 1.0 | 98.4 ± 0.5 | 69.2 ± 0.3 |
| 45 (Example 14) | 85.7 ± 0.5 | 18.3 ± 0.2 | 20.5 ± 0.9 | 9.9 ± 0.8 | 4.6 ± 0.7 | 2.2 ± 1.1 | 98.7 ± 0.6 | 69.6 ± 0.2 |
| 50 | 87.2 ± 0.4 | 18.9 ± 0.4 | 20.0 ± 0.8 | 9.7 ± 0.7 | 3.3 ± 0.8 | 1.7 ± 0.7 | 98.8 ± 0.4 | 69.7 ± 0.2 |
| 55 | 88.8 ± 0.4 | 19.8 ± 0.3 | 19.6 ± 0.7 | 9.4 ± 0.6 | 2.2 ± 0.5 | 1.0 ± 0.4 | 98.9 ± 0.8 | 69.8 ± 0.3 |
| 60 | 89.3 ± 0.5 | 20.4 ± 0.4 | 19.0 ± 0.5 | 9.2 ± 0.4 | 1.0 ± 0.4 | 0.4 ± 0.2 | 98.9 ± 0.7 | 70.0 ± 0.4 |
| 65 | 90.1 ± 0.6 | 20.9 ± 0.5 | 18.5 ± 1.0 | 9.0 ± 0.6 | 0 | 0 | 99.0 ± 0.9 | 70.1 ± 0.4 |
| 70 | 90.2 ± 0.7 | 20.9 ± 0.4 | 18.4 ± 1.2 | 8.9 ± 0.7 | 0 | 0 | 99.1 ± 0.7 | 70.2 ± 0.3 |
| 75 | 90.2 ± 0.5 | 22.4 ± 0.6 | 18.0 ± 1.0 | 8.8 ± 0.5 | 0 | 0 | 96.5 ± 0.5 | 68.8 ± 0.4 |
| 80 | 90.0 ± 0.5 | 25.0 ± 0.3 | 17.8 ± 1.0 | 8.7 ± 0.4 | 0 | 0 | 91.4 ± 0.5 | 66.3 ± 0.2 |

### Example 16: Co-Fractionation Option 3 for κ- and β-Caseins in MCC

A method for co-fractionation of κ- and β-caseins includes the following steps:

(1) MCC powder was taken and reconstituted in ultrapure water to obtain a reconstituted casein solution having a casein mass concentration of 3%.

(2) A 4 M acetic acid solution was added to adjust a pH of the reconstituted casein solution to 5.0, and a resulting solution was equilibrated under stirring (300 rpm) at 25° C. for 90 minutes, and equilibrated under stirring (300 rpm) at 2° C. for 8 hours.

(3) Centrifugation was performed at 2° C. and 3,000 g for 10 minutes to obtain a precipitate enriched with αₛ-casein and a supernatant enriched with κ- and β-caseins.

(4) The supernatant enriched with κ- and β-caseins was collected, a 2 M hydrochloric acid solution was added at 25° C. to adjust a pH of the supernatant to 3.6, and a resulting solution was equilibrated under stirring for 20 minutes after being heated to 50° C., and centrifuged at 10,000 g for 10 minutes after being cooled to 25° C. to obtain a precipitate enriched with κ- and β-caseins and a supernatant depleted in κ- and β-caseins.

The yields and purities of κ-casein and β-casein obtained in step (4) are shown in Table 28. As can be seen from Table 28, the method of Example 16 is capable of effectively co-fractionating κ- and β-caseins.

TABLE 28
Co-fractionation results of κ- and β-caseins in Example 16

| | |
|---|---|
| κ-casein | β-casein |

| Yield (%) | Purity (%) | Yield (%) | Purity (%) |
|---|---|---|---|
| 43.9 ± 0.4 | 14.0 ± 0.1 | 76.5 ± 0.3 | 81.4 ± 0.3 |

### Example 17: Co-Fractionation Option 3: Effect of Different Acidifying Agents

### Regulation of Different Acidifying Agents:

The acetic acid solution in the step (2) of Example 16 was adjusted to be a citric acid solution, or a hydrochloric acid solution. All other steps remained consistent with those in Example 16.

In the acidifying agent regulation solutions, the extraction rates and purities of κ-casein and β-casein obtained in the step (3) are shown in Table 29.

TABLE 29
Co-fractionation results in acidifying agent
regulation solutions in Example 17

| | κ-casein | | β-casein | |
|---|---|---|---|---|
| | Extraction | Purity | Extraction | Purity |
| Acidifying agent | rate (%) | (%) | rate (%) | (%) |
| Acetic acid | 48.4 ± 0.4 | 16.1 ± 0.1 | 76.6 ± 0.3 | 81.9 ± 0.3 |
| (Example 16) | | | | |
| Hydrochloric acid | 42.3 ± 0.3 | 13.6 ± 0.4 | 73.4 ± 0.3 | 81.3 ± 0.5 |
| Citric acid | 23.3 ± 0.3 | 12.2 ± 0.3 | 44.5 ± 0.5 | 80.4 ± 0.2 |

As can be seen from Table 29, an order of the extraction rates of both κ-casein and β-casein is: acetic acid>hydrochloric acid>citric acid; and an order of the purities of κ-casein and β-casein is: acetic acid>hydrochloric acid>citric acid. Therefore, the acetic acid solution is the most preferred acidifying agent for the co-fractionation of κ- and β-caseins.

What is claimed is:

1. A method for co-fractionation of κ- and β-caseins from casein micelles, comprising the following steps:
(1) co-fractionation:
option 1: at 23-27° C., adjusting a pH of a casein solution having a mass concentration of 2-4% to 11.0; subsequently adding a calcium chloride solution until a concentration of added calcium ions reaches 5-65 mM; equilibrating a resulting solution at 10-25° C. for 60-90 minutes to obtain a casein-calcium complexed solution; then subjecting the casein-calcium complexed solution to low-temperature treatment at 0-12° C. for 0-60 minutes, adjusting a pH of the casein-calcium complexed solution to 4.9-5.1 for acid precipitation, and equilibrating a resulting solution at 0-12° C. for 8-24 hours; and performing centrifugation to obtain a precipitate enriched with αₛ-casein and a supernatant enriched with κ- and β-caseins; wherein the pH is adjusted to 4.9-5.1 with a 4 M acetic acid solution, a 4 M lactic acid solution, or a 4 M citric acid solution; or
option 2: at 23-27° C., adjusting a pH of a casein solution having a mass concentration of 2-4% to 4.3, heating a resulting solution to 40-50° C., and equilibrating the solution for 20-40 minutes; then performing centrifugation after cooling to 23-27° C. to obtain a precipitate; collecting the precipitate, placing the precipitate in ultrapure water, and adjusting a pH of the same to 7.5 at 23-27° C., followed by equilibration at 23-27° C. for 60-75 minutes to obtain a reconstituted casein solution, wherein a casein mass concentration is 2-4%; adding a calcium chloride solution to the reconstituted casein solution until a concentration of added calcium ions reaches 45-70 mM, and equilibrating a resulting solution at 23-27° C. for 50-70 minutes; adjusting a pH of the resulting solution to 5.0 for acid precipitation to obtain a solution, equilibrating the solution at 23-27° C. for 60-150 minutes, and subjecting the solution to low-temperature treatment at 0-12° C. for 7-9 hours; and performing centrifugation to obtain a precipitate enriched with αₛ-casein and a supernatant enriched with κ- and β-caseins; wherein the pH is adjusted to 5.0 with a 4 M acetic acid solution; and
(2) collecting the supernatant enriched with κ- and β-caseins, adjusting a pH of the supernatant to 3.4-3.8, allowing precipitation at 45-55° C. for 20-60 min, and performing centrifugation to obtain a precipitate enriched with κ- and β-caseins.

2. The method according to claim 1, wherein in the co-fractionation option 1 of the step (1), the equilibration is an equilibration under stirring at a speed of 200-500 rpm.

3. The method according to claim 1, wherein in the co-fractionation option 1 of the step (1), the mass concentration of the casein solution is 3%.

4. The method according to claim 1, wherein in the co-fractionation option 1 of the step (1), the casein solution is prepared by reconstituting micellar casein concentrate (MCC) powder in water or by diluting a reconstituted MCC solution.

5. The method according to claim 1, wherein in the co-fractionation option 1 of the step (1), the concentration of added calcium ions is 35 mM.

6. The method according to claim 1, wherein in the co-fractionation option 2 of the step (1), the centrifugation is performed at 3.5-4.5° C. and 2,000-10,000 g for 5-15 minutes.

7. The method according to claim 1, wherein in the co-fractionation option 1 of the step (1), the pH is adjusted to 11.0 with a 2 M NaOH solution.

8. The method according to claim 1, wherein in the co-fractionation option 1 of the step (1), the centrifugation is performed at 3.5-4.5° C. and 2,000-10,000 g for 5-15 minutes.

9. The method according to claim 1, wherein in the co-fractionation option 2 of the step (1), the temperature of the low-temperature treatment is 0-2° C.
